SEQUENCE LISTING (78 sequences)

SEQ ID NO: 1 - protein, 234 aa, Artificial Sequence
  Light chain of Fab dimeric molecular complexes pGT821, pGT823, pGT841, pGT842, pGT853, pGT854, pGT855, pGT856, pGT857, pGT858, pGT2008, pGT2009, pGT2010, pGT2011, pGT2012 and pGT2013.
MVLQTQVFISLLLWISGAYGDIQMTQSPSSLSASVGDRVTITCRASQDINNYLSWYQQKPGKAPKLLIYGASNLQSGVPSRFSGSGSGTDFTLTISSLQPEDFAVYYCQQYYGRPTTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 2 - protein, 376 aa, Artificial Sequence
  Heavy chain of dimeric molecular complex pGT821
MKHLWFFLLLVAAPRWVLSQVELVESGGGLVQPGGSLRLSCAASGFTFSSYGMSWVRQAPGKGLEWVSGISSLGSTTYYADSVKGRFTISRDNSKNTLYLQMNSLRAEDTAVYYCARTGSPGTFMHGDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSTKTSGPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVSVNPGK

SEQ ID NO: 3 - protein, 382 aa, Artificial Sequence
  Heavy chain of dimeric molecular complex pGT823
MKHLWFFLLLVAAPRWVLSQVELVESGGGLVQPGGSLRLSCAASGFTFSSYGMSWVRQAPGKGLEWVSGISSLGSTTYYADSVKGRFTISRDNSKNTLYLQMNSLRAEDTAVYYCARTGSPGTFMHGDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSTKTSGPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVSVNPESSRRGGC

SEQ ID NO: 4 - protein, 376 aa, Artificial Sequence
  Heavy chain of dimeric molecular complex pGT841
MKHLWFFLLLVAAPRWVLSQVELVESGGGLVQPGGSLRLSCAASGFTFSSYGMSWVRQAPGKGLEWVSGISSLGSTTYYADSVKGRFTISRDNSKNTLYLQMNSLRAEDTAVYYCARTGSPGTFMHGDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSSDKTHTCPPCPAPELLGGPSTKTSGPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVSVNPGK

SEQ ID NO: 5 - protein, 382 aa, Artificial Sequence
  Heavy chain of dimeric molecular complex pGT842
MKHLWFFLLLVAAPRWVLSQVELVESGGGLVQPGGSLRLSCAASGFTFSSYGMSWVRQAPGKGLEWVSGISSLGSTTYYADSVKGRFTISRDNSKNTLYLQMNSLRAEDTAVYYCARTGSPGTFMHGDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSSDKTHTCPPCPAPELLGGPSTKTSGPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVSVNPESSRRGGC

SEQ ID NO: 6 - protein, 234 aa, Artificial Sequence
  Light chain of dimeric molecular complex pGT843
MVLQTQVFISLLLWISGAYGDIQMTQSPSSLSASVGDRVTITCRASQDINNYLSWYQQKPGKAPKLLIYGASNLQSGVPSRFSGSGSGTDFTLTISSLQPEDFAVYYCQQYYGRPTTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGES

SEQ ID NO: 7 - protein, 376 aa, Artificial Sequence; identical to SEQ ID NO: 2
  Heavy chain of dimeric molecular complex pGT843

SEQ ID NO: 8 - protein, 234 aa, Artificial Sequence; identical to SEQ ID NO: 6
  Light chain of dimeric molecular complex pGT844

SEQ ID NO: 9 - protein, 382 aa, Artificial Sequence; identical to SEQ ID NO: 3
  Heavy chain of dimeric molecular complex pGT844

SEQ ID NO: 10 - protein, 376 aa, Artificial Sequence
  Heavy chain of dimeric molecular complex pGT853
MKHLWFFLLLVAAPRWVLSQVELVESGGGLVQPGGSLRLSCAASGFTFSSYGMSWVRQAPGKGLEWVSGISSLGSTTYYADSVKGRFTISRDNSKNTLYLQMNSLRAEDTAVYYCARTGSPGTFMHGDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSTKTSGPRAAPEVYAFATPEWPGSRDKRTLACLIQNFMPEDISVQWLHNEVQLPDARHSTTQPRKTKGSGFFVFSRLEVTRAEWEQCDEFICRAVHEAASPSQTVQRAVSVNPGK

SEQ ID NO: 11 - protein, 382 aa, Artificial Sequence
  Heavy chain of dimeric molecular complex pGT854
MKHLWFFLLLVAAPRWVLSQVELVESGGGLVQPGGSLRLSCAASGFTFSSYGMSWVRQAPGKGLEWVSGISSLGSTTYYADSVKGRFTISRDNSKNTLYLQMNSLRAEDTAVYYCARTGSPGTFMHGDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSTKTSGPRAAPEVYAFATPEWPGSRDKRTLACLIQNFMPEDISVQWLHNEVQLPDARHSTTQPRKTKGSGFFVFSRLEVTRAEWEQCDEFICRAVHEAASPSQTVQRAVSVNPESSRRGGC

SEQ ID NO: 12 - protein, 376 aa, Artificial Sequence
  Heavy chain of dimeric molecular complex pGT855
MKHLWFFLLLVAAPRWVLSQVELVESGGGLVQPGGSLRLSCAASGFTFSSYGMSWVRQAPGKGLEWVSGISSLGSTTYYADSVKGRFTISRDNSKNTLYLQMNSLRAEDTAVYYCARTGSPGTFMHGDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSTKTSGPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVCRAVSVNPGK

SEQ ID NO: 13 - protein, 382 aa, Artificial Sequence
  Heavy chain of dimeric molecular complex pGT856
MKHLWFFLLLVAAPRWVLSQVELVESGGGLVQPGGSLRLSCAASGFTFSSYGMSWVRQAPGKGLEWVSGISSLGSTTYYADSVKGRFTISRDNSKNTLYLQMNSLRAEDTAVYYCARTGSPGTFMHGDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSTKTSGPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVCRAVSVNPESSRRGGC

SEQ ID NO: 14 - protein, 376 aa, Artificial Sequence
  Heavy chain of dimeric molecular complex pGT857
MKHLWFFLLLVAAPRWVLSQVELVESGGGLVQPGGSLRLSCAASGFTFSSYGMSWVRQAPGKGLEWVSGISSLGSTTYYADSVKGRFTISRDNSKNTLYLQMNSLRAEDTAVYYCARTGSPGTFMHGDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSTKTSGPRAAPEVYAFATPEWPGSRDKRTLACLIQNFMPEDISVQWLHNECQLPDARHSTTQPRKTKGSGFFVFSRLEVTRAEWEQKDEFICRAVHEAASPSQTVQRAVSVNPGK

SEQ ID NO: 15 - protein, 382 aa, Artificial Sequence
  Heavy chain of dimeric molecular complex pGT858
MKHLWFFLLLVAAPRWVLSQVELVESGGGLVQPGGSLRLSCAASGFTFSSYGMSWVRQAPGKGLEWVSGISSLGSTTYYADSVKGRFTISRDNSKNTLYLQMNSLRAEDTAVYYCARTGSPGTFMHGDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSTKTSGPRAAPEVYAFATPEWPGSRDKRTLACLIQNFMPEDISVQWLHNECQLPDARHSTTQPRKTKGSGFFVFSRLEVTRAEWEQKDEFICRAVHEAASPSQTVQRAVSVNPESSRRGGC

SEQ ID NO: 16 - protein, 382 aa, Artificial Sequence
  Heavy chain of dimeric molecular complex pGT2008
MKHLWFFLLLVAAPRWVLSQVELVESGGGLVQPGGSLRLSCAASGFTFSSYGMSWVRQAPGKGLEWVSGISSLGSTTYYADSVKGRFTISRDNSKNTLYLQMNSLRAEDTAVYYCARTGSPGTFMHGDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSTKTSGPRCAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVSVNPESSRRGGC

SEQ ID NO: 17 - protein, 382 aa, Artificial Sequence
  Heavy chain of dimeric molecular complex pGT2009
MKHLWFFLLLVAAPRWVLSQVELVESGGGLVQPGGSLRLSCAASGFTFSSYGMSWVRQAPGKGLEWVSGISSLGSTTYYADSVKGRFTISRDNSKNTLYLQMNSLRAEDTAVYYCARTGSPGTFMHGDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSTKTSGPRAAPEVYAFATPEWPGSRDKRTLACLIQNFMPEDISVQWLHNEVQLPDCRHSTTQPRKTKGSGFFVFSRLEVTRAEWEQKDEFICRAVHEAASPSQTVQRAVSVNPESSRRGGC

SEQ ID NO: 18 - protein, 382 aa, Artificial Sequence
  Heavy chain of dimeric molecular complex pGT2010
MKHLWFFLLLVAAPRWVLSQVELVESGGGLVQPGGSLRLSCAASGFTFSSYGMSWVRQAPGKGLEWVSGISSLGSTTYYADSVKGRFTISRDNSKNTLYLQMNSLRAEDTAVYYCARTGSPGTFMHGDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSTKTSGPRAAPEVYAFATPEWPGSRDKRTLACLIQNFMPEDISVQWLHNEVQLPDARHSTTQPRKTKGSGFFVFSRLEVTRCEWEQKDEFICRAVHEAASPSQTVQRAVSVNPESSRRGGC

SEQ ID NO: 19 - protein, 382 aa, Artificial Sequence
  Heavy chain of dimeric molecular complex pGT2011
MKHLWFFLLLVAAPRWVLSQVELVESGGGLVQPGGSLRLSCAASGFTFSSYGMSWVRQAPGKGLEWVSGISSLGSTTYYADSVKGRFTISRDNSKNTLYLQMNSLRAEDTAVYYCARTGSPGTFMHGDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSTKTSGPRAAPEVYAFATPEWPGSRDKRTLACLIQNFMPEDISVQWLHNEVQLPDARHSTTQPRKTKGSGFFVFSRLEVTRAEWEQKDEFICRAVHEAACPSQTVQRAVSVNPESSRRGGC

SEQ ID NO: 20 - protein, 382 aa, Artificial Sequence
  Heavy chain of dimeric molecular complex pGT2012
MKHLWFFLLLVAAPRWVLSQVELVESGGGLVQPGGSLRLSCAASGFTFSSYGMSWVRQAPGKGLEWVSGISSLGSTTYYADSVKGRFTISRDNSKNTLYLQMNSLRAEDTAVYYCARTGSPGTFMHGDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSTKTSGPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRCVSVNPESSRRGGC

SEQ ID NO: 21 - protein, 382 aa, Artificial Sequence
  Heavy chain of dimeric molecular complex pGT2013
MKHLWFFLLLVAAPRWVLSQVELVESGGGLVQPGGSLRLSCAASGFTFSSYGMSWVRQAPGKGLEWVSGISSLGSTTYYADSVKGRFTISRDNSKNTLYLQMNSLRAEDTAVYYCARTGSPGTFMHGDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSTKTSGPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVCVNPESSRRGGC

SEQ ID NO: 22 - protein, 8 aa, Artificial Sequence
  M2“ extension of IgE CH4
ESSRRGGC

SEQ ID NO: 23 - protein, 8 aa, Artificial Sequence
  Modified M2” extension of IgE CH4
ESSKKGGC

SEQ ID NO: 24 - protein, 8 aa, Artificial Sequence
  Modified M2“ extension of IgE CH4
ESSCRGGC

SEQ ID NO: 25 - protein, 8 aa, Artificial Sequence
  Modified M2” extension of IgE CH4
ESSRCGGC

SEQ ID NO: 26 - protein, 8 aa, Artificial Sequence
  Acidic form of the M2“ extension of IgE CH4
ESSEEGGC

SEQ ID NO: 27 - protein, 8 aa, Artificial Sequence
  Basic form of the M2” extension of IgE CH4
ESSRRGGC

SEQ ID NO: 28 - protein, 110 aa, Artificial Sequence
  IgE CH4 domain
PPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVSVNPGK

SEQ ID NO: 29 - protein, 20 aa, Artificial Sequence
  Eu IgG1 CH2 domain hinge
HTCPPCPAPELLGGPSVFLF

SEQ ID NO: 30 - protein, 20 aa, Artificial Sequence
  Variants of the Eu IgG1 CH2 domain hinge
HTCPPCPAPELLGGPSDSEY

SEQ ID NO: 31 - protein, 20 aa, Artificial Sequence
  Variants of the Eu IgG1 CH2 domain hinge
HTCPPCPAPELLGGPSKSKY

SEQ ID NO: 32 - protein, 20 aa, Artificial Sequence
  Variants of the Eu IgG1 CH2 domain hinge
HTCPPCPAPELLGGPSCSEY

SEQ ID NO: 33 - protein, 20 aa, Artificial Sequence
  Variants of the Eu IgG1 CH2 domain hinge
HTCPPCPAPELLGGPSDSCY

SEQ ID NO: 34 - protein, 20 aa, Artificial Sequence
  Variants of the Eu IgG1 CH2 domain hinge
HTCPPCPAPELLGGPSDEEY

SEQ ID NO: 35 - protein, 20 aa, Artificial Sequence
  Variants of the Eu IgG1 CH2 domain hinge
HTCPPCPAPELLGGPSKRKY

SEQ ID NO: 36 - protein, 20 aa, Artificial Sequence
  Variants of the Eu IgG1 CH2 domain hinge
HTCPPCPAPELLGGPSSESE

SEQ ID NO: 37 - protein, 20 aa, Artificial Sequence
  Variants of the Eu IgG1 CH2 domain hinge
HTCPPCPAPELLGGPSSDSD

SEQ ID NO: 38 - protein, 20 aa, Artificial Sequence
  Variants of the Eu IgG1 CH2 domain hinge
HTCPPCPAPELLGGPSSKSK

SEQ ID NO: 39 - protein, 20 aa, Artificial Sequence
  Variants of the Eu IgG1 CH2 domain hinge
HTCPPCPAPELLGGPSSRSR

SEQ ID NO: 40 - protein, 20 aa, Artificial Sequence
  Variants of the Eu IgG1 CH2 domain hinge
HTCPPCPAPELLGGPSSESY

SEQ ID NO: 41 - protein, 20 aa, Artificial Sequence
  Variants of the Eu IgG1 CH2 domain hinge
HTCPPCPAPELLGGPSSDSY

SEQ ID NO: 42 - protein, 20 aa, Artificial Sequence
  Variants of the Eu IgG1 CH2 domain hinge
HTCPPCPAPELLGGPSSKSY

SEQ ID NO: 43 - protein, 20 aa, Artificial Sequence
  Variants of the Eu IgG1 CH2 domain hinge
HTCPPCPAPELLGGPSSRSY

SEQ ID NO: 44 - protein, 20 aa, Artificial Sequence
  Variants of the Eu IgG1 CH2 domain hinge
HTCPPCPAPELLGGPSDEEY

SEQ ID NO: 45 - protein, 20 aa, Artificial Sequence
  Variants of the Eu IgG1 CH2 domain hinge
HTCPPCPAPELLGGPSDDDY

SEQ ID NO: 46 - protein, 20 aa, Artificial Sequence
  Variants of the Eu IgG1 CH2 domain hinge
HTCPPCPAPELLGGPSDDEY

SEQ ID NO: 47 - protein, 20 aa, Artificial Sequence
  Variants of the Eu IgG1 CH2 domain hinge
HTCPPCPAPELLGGPSDEDY

SEQ ID NO: 48 - protein, 20 aa, Artificial Sequence
  Variants of the Eu IgG1 CH2 domain hinge
HTCPPCPAPELLGGPSEEEY

SEQ ID NO: 49 - protein, 20 aa, Artificial Sequence
  Variants of the Eu IgG1 CH2 domain hinge
HTCPPCPAPELLGGPSEDDY

SEQ ID NO: 50 - protein, 20 aa, Artificial Sequence
  Variants of the Eu IgG1 CH2 domain hinge
HTCPPCPAPELLGGPSEDEY

SEQ ID NO: 51 - protein, 20 aa, Artificial Sequence
  Variants of the Eu IgG1 CH2 domain hinge
HTCPPCPAPELLGGPSEEDY

SEQ ID NO: 52 - protein, 20 aa, Artificial Sequence
  Variants of the Eu IgG1 CH2 domain hinge
HTCPPCPAPELLGGPSRRRY

SEQ ID NO: 53 - protein, 20 aa, Artificial Sequence
  Variants of the Eu IgG1 CH2 domain hinge
HTCPPCPAPELLGGPSRKRY

SEQ ID NO: 54 - protein, 20 aa, Artificial Sequence
  Variants of the Eu IgG1 CH2 domain hinge
HTCPPCPAPELLGGPSRRKY

SEQ ID NO: 55 - protein, 20 aa, Artificial Sequence
  Variants of the Eu IgG1 CH2 domain hinge
HTCPPCPAPELLGGPSRKKY

SEQ ID NO: 56 - protein, 20 aa, Artificial Sequence
  Variants of the Eu IgG1 CH2 domain hinge
HTCPPCPAPELLGGPSKKKY

SEQ ID NO: 57 - protein, 20 aa, Artificial Sequence
  Variants of the Eu IgG1 CH2 domain hinge
HTCPPCPAPELLGGPSKRRY

SEQ ID NO: 58 - protein, 20 aa, Artificial Sequence
  Variants of the Eu IgG1 CH2 domain hinge
HTCPPCPAPELLGGPSKRKY

SEQ ID NO: 59 - protein, 20 aa, Artificial Sequence
  Variants of the Eu IgG1 CH2 domain hinge
HTCPPCPAPELLGGPSKKRY

SEQ ID NO: 60 - protein, 12 aa, Homo sapiens
EPKSCDKTHTCP

SEQ ID NO: 61 - protein, 13 aa, Mus musculus
VPRDCGCKPCICT

SEQ ID NO: 62 - protein, 12 aa, Homo sapiens
ERKCCVECPPCP

SEQ ID NO: 63 - protein, 62 aa, Homo sapiens
ELKTPLGDTTHTCPRCPEPKSCDTPPPCPRCPEPKSCDTPPPCPRCPEPKSCDTPPPCPRCP

SEQ ID NO: 64 - protein, 12 aa, Homo sapiens
ESKYGPPCPSCP

SEQ ID NO: 65 - protein, 58 aa, Homo sapiens
SPKAQASSVPTAQPQAEGSLAKATTAPATTRNTGRGGEEKKKEKEKEEQEERETKTPE

SEQ ID NO: 66 - protein, 18 aa, Homo sapiens
PSTPPTPSPSTPPTPSPS

SEQ ID NO: 67 - protein, 5 aa, Homo sapiens
PPPPP

SEQ ID NO: 68 - DNA, 42 bp, Artificial Sequence
  primer
ggataaaaaagtggaaccgaagagctccgataaaactcacac

SEQ ID NO: 69 - DNA, 27 bp, Artificial Sequence
  primer
caggggagagtcttaagcggccgcgcc

SEQ ID NO: 70 - DNA, 34 bp, Artificial Sequence
  primer
gctacacaacgagtgccagctgccggacgcccgg

SEQ ID NO: 71 - DNA, 40 bp, Artificial Sequence
  primer
gggccgaatgggagcagtgcgatgaattcatctgccgtgc

SEQ ID NO: 72 - DNA, 40 bp, Artificial Sequence
  primer
cccctcacagaccgtctgcagagcggtgtctgtaaatccc

SEQ ID NO: 73 - DNA, 31 bp, Artificial Sequence
  primer
gaccagcggcccacggtgtgccccggaagtc

SEQ ID NO: 74 - protein, 35 aa, Artificial Sequence
  primer
GCAGCTCCCGGACTGCAGACACAGCACGACGCAGC

SEQ ID NO: 75 - DNA, 33 bp, Artificial Sequence
  primer
gccgcctggaggtcaccaggtgcgaatgggagc

SEQ ID NO: 76 - DNA, 30 bp, Artificial Sequence
  primer
gtccatgaggcagcatgcccctcacagacc

SEQ ID NO: 77 - DNA, 39 bp, Artificial Sequence
  primer
cctcacagaccgtccagcgctgcgtgtctgtaaatcccg

SEQ ID NO: 78 - DNA, 34 bp, Artificial Sequence
  primer
ccagcgagcggtgtgtgttaaccccgagtcatcg

(19) United States
(12) Patent Application Publication
(10) Pub. No.: US 2013/0259806 A1
(43) Pub. Date: Oct. 3, 2013

(54) DIMERIC MOLECULAR COMPLEXES WITH FREE CYSTEINE RESIDUES AND CONJUGATES THEREOF

(71) Applicants: David Light, San Mateo, CA (US); Paul Szymanski, San Francisco, CA (US); Marian Seto, Orinda, CA (US); Aline Cornelius, Berkeley, CA (US)

(72) Inventors: David Light, San Mateo, CA (US); Paul Szymanski, San Francisco, CA (US); Marian Seto, Orinda, CA (US); Aline Cornelius, Berkeley, CA (US)

(21) Appl. No.: 13/988,133

(22) PCT Filed: Dec. 7, 2011
(86) PCT No.: PCT/US2011/063748

§ 371 Date: May 17, 2013

Related U.S. Application Data
(60) Provisional application No. 61/421,418, filed on Dec. 9, 2010.

(51) Int. Cl. C07K 16/00

(52) U.S. Cl. CPC C07K 16/00
USPC 424/9.1; 530/387.3; 536/23.4; 435/320.1; 435/252.3; 435/254.2; 435/328; 435/419; 530/391.7; 530/391.3; 424/178.1

(57) ABSTRACT

The invention relates generally to dimeric molecular complexes comprising two fusion proteins. Each fusion protein comprises a biological effector moiety, a polypeptide spacer sequence, and an IgE CH4 dimerization domain. The dimeric molecular complexes may be conjugated, at a defined site, to other molecules including drug moieties, cytotoxic agents, labels (such as detectable labels), or biocompatible polymers.

### CROSS-REFERENCE TO RELATED APPLICATION

This application claims the benefit of U.S. Provisional Patent Application Ser. No. 61/421,418 filed Dec. 9, 2010, which is a U.S. national stage application of International Application No. PCT/US2011/063748 filed Dec. 7, 2011, the entire contents of which are incorporated herein by reference.

### SUBMISSION OF SEQUENCE LISTING

The Sequence Listing associated with this application is filed in electronic format via EFS-Web and hereby incorporated by reference into the specification in its entirety. The name of the text file containing the Sequence Listing is Sequence_List_{—}07430_{—}00191_US_BHC105016. The size of the text file is 82 KB, and the text file was created on Dec. 3, 2010.

### FIELD OF THE INVENTION

The invention relates generally to dimeric molecular complexes comprising two fusion proteins. Each fusion protein comprises a biological effector moiety, a polypeptide spacer sequence, and an IgE CH4 dimerization domain. The dimeric molecular complexes may be conjugated, at a defined site, to other molecules including drug moieties, cytotoxic agents, labels (such as detectable labels), or biocompatible polymers. Related formulations of the conjugates and methods of administration thereof for diagnostic and therapeutic uses are also provided.

### BACKGROUND OF THE INVENTION

Many medical uses benefit from targeted delivery of a particular molecule to a specific biological site. For example, antibody-drug conjugates specific for tumor-associated antigens are known for the delivery of cytotoxic agents to kill or inhibit the growth of tumor cells (Wu et al., 2005, Nature Biotech. 23(9): 1137-1146). Conjugation of antibodies to radionuclides may also be used for the detection and imaging of tumors. (Olafsen et al., 2004, Protein Eng. Des. Sel. 17(1): 21-27). A wide range of antibodies and antibody fragments have been developed for tumor cell targeting (WO2007/137760; Song et al., 2006, J. Microbiol. Biotechnol. 16(7): 1104-1110; Wu et al., 2001, Protein Eng. 14(12): 1025-1033). Antibodies have also been useful for localized delivery of immunological agents, such as pharmaceutical compounds to treat immunological disorders such as arthritis, or localized delivery of pharmaceutical agents that treat angiogenic disorders.

U.S. Pat. No. 7,521,541 (Eigenbrot et al.) describes modifying antibodies to replace one or more amino acids with a free cysteine residue. The modified antibody is reacted with a thiol-reactive reagent that is attached to or is a drug or a detectable label, for example.

Dimeric molecular complexes comprising two fusion proteins have been shown to have improved binding activity compared to that of the Fab fragment. WO 2007/137760 (published Dec. 6, 2007) discloses such complexes containing two fusion proteins. In some embodiments, the fusion protein comprises from its N to C terminus (a) a biological effector moiety, (b) a hinge region of an IgG molecule and (c) a CH4 dimerization domain of an IgE molecule. WO 2007/137760 discloses modification to the hinge region to increase the ability to conjugate various molecules to the dimeric molecular complex. These modifications included altering the hinge region to provide additional lysine residues for conjugation. WO 2007/137760 describes the biodistribution of three dimeric antibody formats comprising the CH4 domain from IgE and the antibody L19, which specifically targets the extra domain B (ED-B) of fibronectin localized to the tumor vasculature (Borsi et al., 2002, Int. J. Cancer 102: 75-85). The ¹²⁵I-radiolabelled 50 kDa “small immunoprotein” (SIP) format of the anti-ED-B fibronectin antibody achieved the highest tumor to blood ratio when compared to the diabody and IgG formats. In a subsequent study, the ^{99m}Tc-radiolabelled diabody format of L19 antibody was shown to efficiently allow scintigraphic imaging of F9 tetracarcinoma tumors in mice (Berndorff et al., 2006, J. Nucl. Med. 47: 1707-1716). The biodistribution and PET imaging properties of [⁸⁶Y] and [¹¹¹IN] anti-mindin engineered antibody fragments, including the IgE-CH4 miniantibody, were shown to compare favorably with the IgG format (WO2007/137760). The antibody-related dimeric molecular complexes were described as having superior pharmacokinetic and biodistribution profiles when compared to those of monomeric single chain antibodies, diabodies, or full-sized dimeric IgGs.

There remains a strong need for effective targeted delivery of drugs, detection labels, and toxins to specific biological sites, such as for the treatment of cancer, immunological disorders, and angiogenic disorders, and for imaging targeted areas of the body. Furthermore, there remains a need for uniform conjugates of a targeting entity and a drug, cytotoxic agent, biocompatible polymer or label in which the conjugation is site-specific at one location on the targeting entity.

### SUMMARY OF THE INVENTION

It is an object of the present invention to provide a dimeric molecular complex comprising a first and a second fusion protein, wherein each fusion protein comprises from its N to C terminus: (a) a biological effector moiety, (b) a polypeptide spacer sequence; and (c) a CH4 dimerization domain of an IgE molecule covalently bound to the polypeptide spacer sequence. The biological effector moiety is (1) a single chain antibody; (2) an Fab fragment; (3) an extracellular domain of a type I membrane receptor; (4) a cytokine; (5) a chemokine; (6) an enzyme; or (7) a toxin. The molecular complex comprises at least one disulfide bond between a cysteine residue in the first fusion protein and a cysteine residue in the second fusion protein, and at least one of the fusion proteins comprises one or more free cysteine amino acid residues. At least one of the fusion proteins has a biological effector moiety that is a targeting biological effector moiety selected from the group consisting of (1) a single chain antibody; (2) an Fab fragment; (3) an extracellular domain of a type I membrane receptor; (4) a cytokine; (5) a chemokine; and (6) an enzyme.

It is another object of the present invention to provide a dimeric molecular complex comprising a first and a second fusion protein, wherein each fusion protein comprises from its N to C terminus: (a) an IgE CH4 dimerization domain with a C terminal extension comprised of an M2″ extension of an IgE CH4 splice variant, (b) an amino acid spacer which is covalently bound to the IgE CH4 dimerization domain, and (c) an extracellular domain of a type II membrane receptor. The molecular complex comprises at least one disulfide bond between a cysteine residue in the first fusion protein and a cysteine residue in the second fusion protein, and at least one of the fusion proteins comprises one or more free cysteine amino acid residues.

It is yet another object of the present invention to provide a nucleic acid molecule which encodes a fusion protein comprising from N to C terminus: (a) a biological effector moiety, (b) a polypeptide spacer sequence; and (c) a CH4 dimerization domain of an IgE molecule covalently bound to the polypeptide spacer sequence. The biological effector moiety is: (1) a single chain antibody, (2) an Fab fragment, (3) an extracellular domain of a type I membrane receptor, (4) a cytokine, (5) a chemokine, (6) an enzyme, (7) a toxin and the fusion protein comprises one or more free cysteine amino acid residues.

In one aspect of the invention there is provided a conjugate comprising a dimeric molecular complex coupled to a drug moiety, cytotoxic agent, label, or biocompatible polymer as shown in (I):

D-Lₐ-M (I)

D is the dimeric molecular complex, L is a linker covalently attached to D at a free cysteine on D, M is a conjugate molecule selected from a drug moiety, cytotoxic agent, label, or biocompatible polymer, and a is 0 or 1. The dimeric molecular complex comprises a first and a second fusion protein, wherein each fusion protein comprises from its N to C terminus: (a) a biological effector moiety, (b) a polypeptide spacer sequence, and (c) a CH4 dimerization domain of an IgE molecule covalently bound to the polypeptide spacer sequence. The biological effector moiety is: (1) a single chain antibody, (2) an Fab fragment, (3) an extracellular domain of a type I membrane receptor, (4) a cytokine, (5) a chemokine, (6) an enzyme or (7) a toxin. At least one of the fusion proteins has a biological effector moiety that is a targeting biological effector moiety that is: (1) a single chain antibody, (2) an Fab fragment, (3) an extracellular domain of a type I membrane receptor, or (4) an enzyme. The molecular complex comprises at least one disulfide bond between a cysteine residue in the first fusion protein and a cysteine residue in the second fusion protein, and at least one of the fusion proteins comprises one or more free cysteine amino acid residues that reacts with the linker to form the conjugate or, if a is 0, reacts with the conjugate molecule to form the conjugate.

The invention also relates to methods of making the conjugate and pharmaceutical compositions comprising the conjugate.

In one embodiment, the invention is directed to a method of killing or inhibiting the proliferation of tumor cells or cancer cells comprising administering to a patient in need thereof an effective amount of the composition comprising the conjugate. In this embodiment, the dimeric molecular complex of the conjugate has at least one biological effector moiety that comprises an antigen binding site for a tumor-associated antigen and the conjugate comprises a cytotoxic agent or a drug that is a chemotherapeutic agent.

In another embodiment, the invention provides a method of imaging a target area of a body in a patient in need thereof comprising administering to a patient a conjugate in an amount sufficient to provide a detectable signal at the target area and detecting the signal. In this embodiment, at least one of the biological effector moieties of the first and second fusion proteins targets a specific cell type or tissue in need of imaging and the conjugate comprises a detectable label.

### DESCRIPTION OF THE FIGURES

FIGS. 1A, 1B and 1C are schematic diagrams of specific Fab dimeric molecular complexes. FIG. 1A shows the complex with (pGT823) and without (pGT821) an eight-residue C-terminal sequence from a natural splice variant of IgE called the M2″ extension. These two complexes were used as the framework for experiments with cysteine modifications. FIG. 1B shows complexes with a free cysteine in the CL domain of the light chain (pGT841 and pGT842). FIG. 1C shows complex with a free cysteine in the CH1 domain of the heavy chain (pGT843 and pGT844).

FIG. 2 is a three-dimensional model of the IgE CH4 domain of the Fab dimer with the location of free cysteines indicated. Only the CH4 domain from IgE of the dimeric antibody formats is shown. In the side view (A), the amino termini (N-termini) and carboxyl termini (C-termini) of the domain are indicated. In the top view (B), the view has the hinge attachment site (N-terminus) facing out of the page. The peptide chains are represented by a ribbon structure, the side chains of the mutated surface residues are shown as CPK models and the locations of the disulfide bonds formed by the native Cys in each CH4 domain (Cys464 and Cys524) are shown as ball and stick models. Both CH4 domains of the dimeric complex are shown, one in dark grey (left) and one in light grey (right). The first three residues mutated to Cys (Gln 538, Lys 519, Val 483) are indicated and additional promising positions for Cys addition are also shown (Lys435, Gln484, Gln535, Arg457).

FIG. 3 shows SDS-PAGE of Fab dimers with free cysteine residues in the Fab and IgE CH4 domains. Lane 1: pGT823. Lane 2: pGT842. Lane 3: pGT844. Lane 4: pGT854. Lane 5: pGT856. LHHL: Complete Fab dimer with two Light and two Heavy chains. HH: Partial species with two Heavy chains. HL: Partial species with one Heavy and one Light chain. LL: Partial species with two Light chains. H: Free Heavy chain. L: Free Light chain.

FIG. 4 shows size-exclusion chromatography (SEC) of Fab dimers with free cysteine residues in the Fab and IgE CH4 domain.

FIG. 5 shows SDS-PAGE of pGT2008-2013 free cysteine variants. Lane 1: pGT823. Lane 2: pGT2008. Lane 3: pGT2009. Lane 4: pGT2010. Lane 5: pGT2011. Lane 6: pGT2012. Lane 7: pGT2013. LHHL: Complete Fab dimer with two Light and two Heavy chains. HH: Partial species with two Heavy chains. HL: Partial species with one Heavy and one Light chain. LL: Partial species with two Light chains. H: Free Heavy chain. L: Free Light chain.

FIG. 6 shows SEC of Fab dimers with free cysteine residues in the IgE CH4 domain.

FIG. 7 shows the dose dependent binding of the Fab dimers comprised of free cysteine residues in the Fab or IgE CH4 domains in an ELISA with MN antigen.

FIG. 8 shows the dose dependent binding of the Fab dimers comprised of free cysteine residues in the IgE CH4 domain in an ELISA with MN antigen.

FIG. 9 shows amino acid sequences of Fab dimeric molecular complexes. Light chain sequences are identical for all plasmids except pGT843 and 844. The CH4 IgE M2″ extension is underlined. Mutations are indicated in bold underline.

FIG. 10 shows amino acid sequences of M2″ extensions, hinge regions, spacer sequences and IgE CH4 domain.

### DESCRIPTION OF THE PREFERRED EMBODIMENTS

The invention provides for conjugates for targeted delivery of the conjugate to a particular biological tissue, cell type, molecule, or location in the mammalian body. The conjugates are characterized by site-specific attachment of a conjugate molecule to a dimeric molecular complex.

### Dimeric Molecular Complex

The dimeric molecular complex comprises a first and a second fusion protein, wherein each fusion protein comprises, in N-terminal to C-terminal direction: (a) a biological effector moiety, (b) a polypeptide spacer sequence, and (c) a CH4 dimerization domain of an IgE molecule covalently attached to the polypeptide spacer sequence. In one embodiment, the biological effector moiety is covalently attached directly to the polypeptide spacer sequence and the polypeptide spacer sequence is covalently attached directly to the CH4 dimerization domain of human IgE.

### Biological Effector Moieties

A “biological effector moiety” is a polypeptide which comprises the biologically active portion of molecules such as antibodies, type I and type II membrane receptors, cytokines, enzymes, and the like. Useful biological effector moieties include the biologically active portion of molecules such as single chain antibodies, Fab fragments, extracellular domains of type I or type II membrane receptors, cytokines (including chemokines), active site domains of enzymes, protein hormones, and peptide effector molecules.

At least one of the first and second fusion proteins has a biological effector moiety that is a targeting biological effector moiety such that it provides for a specific targeting of the complex or conjugate to a particular tissue, cell type, molecule or biological location in the mammalian body or in vitro. The targeting biological effector moiety may be the biologically active portion of a molecule such as an antibody, such as a single chain antibody or an Fab fragment, an extracellular domain of a type I membrane receptor, or an enzyme. In one embodiment the targeting moiety is an Fab fragment.

The biological effector moieties in each of the two fusion proteins which comprise a dimeric molecular complex can be identical or can be moieties with two different functions (e.g., an antigen binding site and a toxin). Biological effector moieties which have a useful therapeutic or tissue specific targeting function are especially useful.

Dimeric molecular complexes are stable in vivo and bind to a target of interest with an affinity similar to that of the native molecule from which the biological effector moiety is derived.

scFv and Fab Dimers

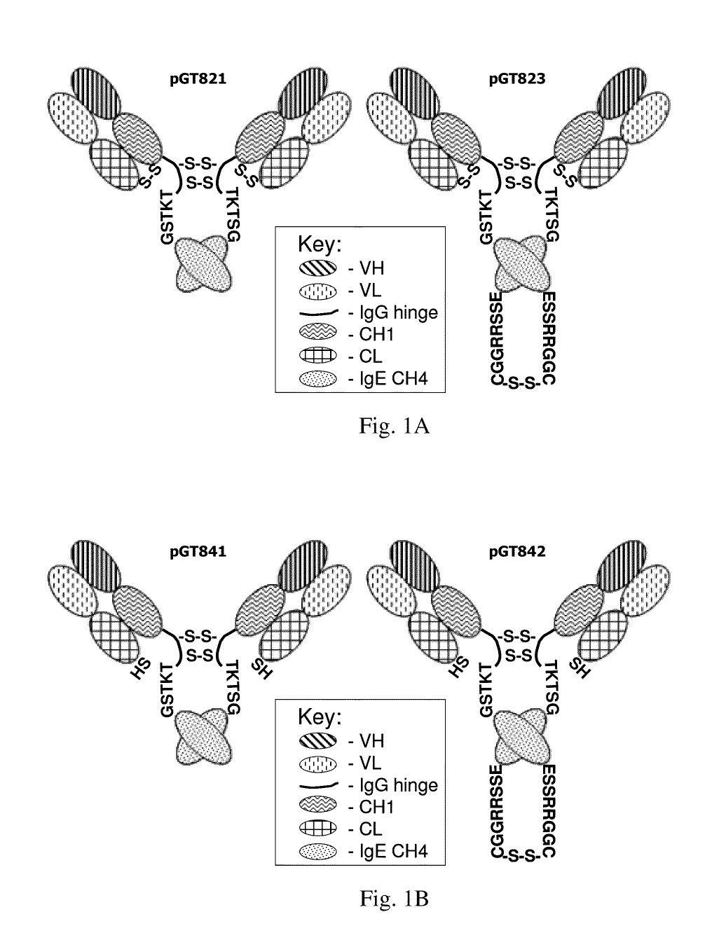

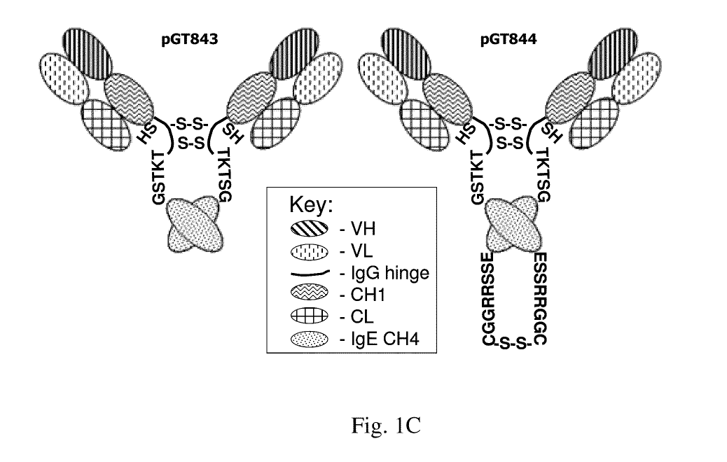

The dimeric molecular complexes of the invention can be formed using fusion proteins comprising the biologically active portion of antibodies, such as the antigen binding or variable region thereof, as the biological effector moieties. In some embodiments, the biological effector moieties of each fusion protein comprise antigen binding sites. The antigen binding sites can be provided, for example, by a single-chain antibody (scFv) or an Fab fragment. The resulting dimeric molecular complex will comprise two antigen binding sites, which can be of the same or different specificities (i.e. forming monospecific or bispecific dimeric molecular complexes, respectively). For example, a monospecific dimeric molecular complex may comprise fusion proteins in which the biological effector moiety of each fusion protein is an scFV. A monospecific dimeric molecular complex may also comprise fusion proteins in which the biological effector moiety of each fusion protein is the same Fab fragment (i.e. a Fab homodimer) as shown schematically in FIG. 1. A bispecific dimeric molecular complex may comprise fusion proteins in which the biological effector moiety of each fusion protein is a different Fab fragment (i.e. a Fab heterodimer).

The antibody-related dimeric molecular complexes of the invention can be used diagnostically and therapeutically. For example, a dimeric molecular complex can be used for in vivo imaging, such as PET imaging, or for in vitro diagnostics. When conjugated to a therapeutic molecule, a dimeric molecular complex can be used to target the therapeutic molecule to a particular target.

In some embodiments, the antigen binding site may be designed to target the dimeric molecular complex to a specific cell type. The antigen binding sites may bind a range of different antigens including cell surface markers and other cell surface proteins such as CD4, CD20, CD22, CD33, CD52, CD66, CD74, CD138, ErbB2, E-selectin, MUC16 and EGF-receptor. The antigen binding sites may also bind cyotokines or other soluble proteins such as VEGF, IL-2, IL-15 and TNFα.

In one embodiment, the two fusion proteins within the dimeric complex each comprise single chain antibodies linked to the IgE CH4 domain via the polypeptide spacer sequence and further comprise N-terminal amino acids including a portion of the first β sheet of the CH2 domain of human IgG1. The benefit of this attachment is that the disulfide bonds in the polypeptide spacer sequences between the two fusion proteins will be located in the middle of the dimeric molecular complex, thus stabilizing the complex. Such dimeric molecular complexes typically have a molecular weight between 50 kD and 150 kD.

The dimeric molecular complexes of the invention can also be made using Fab heavy chain fragments instead of single chain antibodies as the biological effector molecules in the fusion proteins which make up the dimeric complex. When forming a dimeric molecular complex comprising Fab fragments, the CH1 domain of the Fab fragment can be directly linked to the polypeptide spacer sequence, and the polypeptide spacer sequence may consist of an IgG hinge. In this way, the amount of non-native, immunogenic sequences in the molecule may be reduced.

A Fab dimer may be constructed by eliminating the Fc region from an IgG antibody, producing an antibody fragment which has the same affinity as an IgG but a lower molecular weight. The lower molecular weight of such a dimer can result in increased tumor penetration. Moreover, such Fab dimers will not bind to cellular Fc receptors (FcR), thus reducing unwanted FcR-related interactions. For example, a dimeric molecular complex using such Fab dimers can be used to make cytotoxic agent conjugates which will not bind cells with FcγR or the neonatal Fc receptor (FcRn).

When the Fc binding domain is included in an Fc fusion protein, the dimeric molecular complex generated can bind not only to the Fc receptor but also to other proteins (e.g., Fcyllla, Fcvllb, Clq, etc.) and can trigger the effector functions of these molecules. Binding to Fc receptors permits an antibody cytotoxic agent conjugate to be directed to cells that express the Fc receptor. In vivo properties of an antibody that may be mediated by Fc receptor interaction include antigen dependent cellular cytotoxicity (ADCC), phagocytosis, complement dependent cytotoxicity, increased serum half-life, and decreased clearance, etc. The terms “Fc fusion protein1” or “Fc fusion” are widely used to refer to the practice of dimerizing proteins using the IgG hinge and the CH2 and CH3 domains. Such “Fc fusions” retain the ability to bind Fc receptors.

A variety of biological effector moieties other than antibody-related molecules can be used in the fusion proteins of the dimeric molecular complexes of the invention. Examples of these moieties are shown below. Depending on the intended use, dimeric molecular complexes may be comprised of fusion proteins where the two biological effector moieties are the same or different (i.e. homodimers or heterodimers, respectively). Heterodimers are stabilized in a manner similar to that described above.

### Extracellular Domains of Type I Membrane Receptors

A dimeric molecular complex may be comprised of fusion proteins in which the biological effector moiety is an extracellular domain of a type I membrane receptor. Such complexes are useful as biological effectors or to bind ligands. Type I membrane receptors useful in the invention include, but are not limited to, TNF receptors, ephrins, Ephs, VEGF receptors, IGF receptors, thrombospondin, thrombomodulin, PDGF receptors, IL-2R, TCR complex components, EGF receptors, TGF receptors, tissue factor, growth factor receptors, HGH receptor, IFN receptors, HER2, and insulin receptor.

### Cytokines

In some embodiments, the biological effector moiety is a cytokine useful for modulating biological responses of cells. Cytokines useful in the invention include lymphokines such as macrophage activating factor (MAF), macrophage migration inhibition factor (MMIF), leukocyte migration inhibition factor (MCF), leukocyte migration inhibition factor (LMIF), a histamine releasing factor (HRF), or transfer factor (TF). Tumor necrosis factors, such as TNF-α (cachectin) and TNF-β (lymphotoxin) can also be cytokine biological effector moieties. Interleukins, such as IL-1, IL-2, IL-3, IL-4, IL-5, IL-6, IL-7, IL-S, IL-9, IL-10, IL-11, IL-12, IL-13, IL-14, and IL-15, IL-17, can be cytokine biological effector moieties. Interferons, such as IFN-α, IFN-β, IFN-γ, IFN-ω, and IF-τ, can also be cytokine biological effector moieties.

Other useful cytokines include colony stimulating factors, chemokines, and stress proteins. Examples of colony stimulating factors include granulocyte colony stimulating factor (G-CSF), granulocyte macrophage colony stimulating factor (GM-CSF), macrophage colony stimulating factor (M-CSF), and multi-CSF (IL-3).

### Chemokines

Examples of α-chemokines include IL-8, NAP-2 (neutrophil activating protein 2), PF-4 (platelet factor 4), and βTG (β-thromboglobulin). β-Chemokines include MCP-1 (monocytes chemoattractant protein 1), MCP-3, MIP-1α (macrophage inflammatory protein 1α), MIP-1β, and RANTES (“Regulated upon Activation Normal T Expressed and presumably Secreted chemokine”). Other useful chemokines include, e.g., CCL chemokines; CXC (SCYB) chemokines or CX3C chemokines; XC chemokines; and CC chemokines, such as CCL2, CCL7, CCL11, CCL8, CCL13, CCL1, CCL5, CCL16, CCL14, CCL15, CCL23, CCL18, CCL3 and CCL4.

### Enzymes

In other embodiments, a biological effector moiety is an enzyme or a biologically active portion of an enzyme capable of selective binding to a natural substrate of the enzyme (collectively referred to herein as “enzyme”), e.g., a proteolytic enzyme (an amino peptidase, an aspartyl protease, a serine protease, a metallo-protease, a cysteinyl protease, pepsin, trypsin, thrombin, lysozyme, Factor VII, Factor X, or Factor IX). Other enzymes, such as glycosidases, esterases, hydrolases, nucleases, synthases, isomerases, polymerases, kinases, phosphatases, reductases, including oxido-reductases, transferases, ligases, restriction enzymes, amidases, ATPases, carbohydrases, lipases, cellulases, dehydrogenases, and oxidases also can be used.

### Toxins

Therapeutically useful protein toxins can be used as biological effector moieties in the fusion proteins comprising the dimeric molecular complexes of the invention. There are numerous examples of such protein toxins, well known to those skilled in the art, such as the bacterial protein toxins Pseudomonas exotoxin A and diphtheria toxin, and the plant protein toxins ricin, abrin, modeccin, saporin, and gelonin.

### Extracellular Domains of Type II Membrane Receptors

A dimeric molecular complex also can be constructed wherein the biological effector moiety is an extracellular domain of a type II membrane receptor. In this case, however, each of the two fusion proteins comprises, from N to C terminus, (a) an IgE CH4 dimerization domain with a C terminal extension comprised of an M2″ extension of an IgE CH4 splice variant, (b) an amino acid spacer (preferably 3-10 residues in length) which is covalently bound to the IgE CH4 dimerization domain and (c) an extracellular domain of the type II membrane receptor. In one embodiment, the M2″ extension is the amino acid sequence of SEQ ID NO: 22. In other embodiments, the sequence of the M2″ extension can be modified to provide additional sites for chemical modification (e.g. ESSKKGGC (SEQ ID NO: 23), ESSCRGGC (SEQ ID NO: 24) or ESSRCGGC (SEQ ID NO: 25)).

Type II membrane receptors comprise only about 5% of transmembrane proteins, but include members with important biological effector functions such as hepsin protease, ectodysplasin, collagenous membrane proteins, macrophage scavenger receptors, MARCO protein, TNF ligand-like proteins, asialoglycoprotein receptors, lymphocyte IgE receptor, Kupffer cell receptor, NKG2, NKR-P1, Ly-49, CD69, CD72, LyB-2, collectins and CLEC5A.

### Polypeptide Spacer Sequence

As used herein, the term “polypeptide spacer sequence” refers to a peptide or polypeptide sequence (e.g., a synthetic peptide or polypeptide sequence) which connects two domains in an amino acid sequence of a polypeptide chain, such as by covalent attachment. For example, a polypeptide spacer sequence may be used to connect a biological effector moiety to an IgE CH4 domain in a fusion protein. Polypeptide spacer sequences may provide flexibility to the fusion protein and allow the biological effector moiety greater access to its ligand. In one embodiment, the C-terminus of the biological effector moiety is covalently attached directly to the polypeptide spacer sequence. The polypeptide spacer sequence does not comprise immunoglobulin Fc domains, such as CH2, CH3 or CH4 domains, or derivatives or fragments thereof.

The polypeptide spacer sequence may comprise an IgG hinge. An IgG hinge may be, for example, an IgG₁ hinge, an IgG₂ hinge, an IgG₃ hinge or an IgG₄ hinge. The term “IgG₁ hinge” is used to describe a hinge region from a native IgG₁, such as a native human, mouse, rabbit, etc. IgG₁, derivatives of one of the naturally occurring IgG₁ hinges, such as derivatives that have at least 95, 90, 85, 80, 75, 70, or 65% amino acid identity with a native IgG₁ hinge and retain essential functional characteristics of the IgG₁ hinge, such as to enable the biological effector moiety to orient in different directions and enable antigen binding, or other specific ligand binding. In other embodiments, the polypeptide spacer sequence may comprise hinge regions or spacer regions from other immunoglobulin classes (e.g. IgM, IgD, IgA and IgE), or derivatives of these regions. Examples of these regions include, but are not limited to, the IgD hinge (SEQ ID NO: 65), the IgA₁ hinge (SEQ ID NO: 66) and the IgA₂ hinge (SEQ ID NO: 67). Similarly as with the term “IgG₁ hinge,” the hinges or spacer regions from these other IgGs or immunoglobulin classes include derivatives of one of the naturally occurring IgG hinges or spacer regions, such as derivatives that have at least 95, 90, 85, 80, 75, 70, or 65% amino acid identity with a native IgG hinge or spacer region and retain essential functional characteristics of the hinge, such as to allowing the biological effector moiety to orient in different directions and enabling antigen binding, or other specific ligand binding.

The polypeptide spacer sequence may contain, comprise, consist essentially of, or consist of an IgG₁ hinge having amino acid residues 223 to 243 from within the first beta strand of the IgG₁ CH2 domain, where the numbering is based on the sequence of the Eu IgG₁ heavy chain as described in Edelman et al. (1969) Proc. Natl. Acad. Science USA, 63, pp. 78-85. Amino acid residues 223 to 243 of the Eu IgG₁ heavy chain are shown in SEQ ID NO: 29. In some embodiments, the tetrapeptide at positions 240-243 is VFLF (as shown in SEQ ID NO: 29), or is replaced with a tetrapeptide selected from the group consisting of DSEY (SEQ ID NO: 30), KSKY (SEQ ID NO: 31), DEEY (SEQ ID NO: 34) and KRKY (SEQ ID NO: 35). In certain embodiments, the tetrapeptide is DSEY (SEQ ID NO: 30) or KSKY (SEQ ID NO: 31). In other embodiments, the polypeptide spacer sequence may contain, comprise, consist essentially of, or consist of an amino acid sequence selected from the group consisting of SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, and SEQ ID NO: 59. The polypeptide spacer sequence may comprise an amino acid sequence having at least 95, 90, 85, 80, 75, 70, 65, 60, 55, or 55% identity to the amino acid sequence of SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, or SEQ ID NO: 59.

In another embodiment, the polypeptide spacer sequence comprises a polypeptide chain of 1-200, 1-150, 10-150, 1-100, 10-100, 1-90, 10-90, 1-80, 10-80, 1-70, 10-70, 1-60, 10-60, 1-50, 10-50, 1-40, 10-40, 1-30, 10-30, 1-20, 10-20, 1-10, 5-10, 5-200, 10-200, 20-200, 30-200, 40-200, 50-200, 60-200, 70-200, 80-200, 90-200, 100-200, 110-200, 120-200, 130-200, 140-200, 150-200, 160-200, 170-200, 180-200, or 190-200 amino acids in length that is flexible and allows the biological effector moiety to orient in different directions and enables antigen binding, or other specific ligand binding. In such embodiments the polypeptide spacer sequence may or may not contain an IgG hinge region as discussed above, or a spacer region from another immunoglobulin class. In other embodiments, the spacer sequence is an amino acid spacer having sufficient length to allow flexibility as is known in the art. The amino acid spacer may be a poly-alanine or poly-glycine amino acid sequence, and may include other amino acids, including proline, alanine, serine, threonine and aspartate. In some embodiments, each of the fusion proteins of the dimeric molecular complex comprises the same spacer sequence. In other embodiments, each fusion protein of the dimeric molecular complex comprises a different spacer sequence.

### Dimerization

The dimeric molecular complex used in the present invention exploits the self-dimerization properties of the IgE CH4 domain. The CH4 domain allows dimerization of IgE molecules (Batista et al., 1996, PNAS 93: 3399-3404), while the IgE binding sites for the Fc receptor FcεRI reside in the CH3 domain (Turner and Kinet, 1999, Nature 402S: B24-B30; Vangelista et al., 1999, J. Clin. Invest. 103: 1571-1578; Garman et al., 2000, Nature 406: 259-266). Therefore fusion proteins comprising IgE CH4 domains in accordance with embodiments of the present invention do not activate signaling leading to hypersensitivity reactions.

In addition to the dimerization of the two fusion proteins provided by the IgE CH4 domain, Applicants have found it helpful to further stabilize the dimeric molecular complex. M2″ extensions of IgE CH4 splice variants can be added at the C-termini of the fusion proteins comprising the dimeric molecular complex. In one embodiment, an acidic form of the M2″ extension (ESSEEGGC (SEQ ID NO: 26)) is added to the C-terminus of each fusion protein. In another embodiment, an M2″ extension containing basic amino acids (ESSRRGGC (SEQ ID NO: 27) is added to the C-terminus of each fusion protein. Other useful M2″ extensions of IgE splice variants are SEQ ID NO: 22, SEQ ID NO: 23, SEQ ID NO: 24 and SEQ ID NO: 25. In a further embodiment, the acidic form of the M2″ extension is added to one fusion protein of a dimeric molecular complex and the basic form of the M2″ extension is added to the other fusion protein of the complex.

The dimeric molecular complex is also characterized by having at least one disulfide bond between cysteine residues from each of the first and second fusion proteins. In one embodiment, the complex is characterized by having 1, 2, 3, 4, 5 or 6 disulfide bonds between the first and second fusion proteins. The disulfide bonds may be located at various attachment sites within the fusion proteins as long as the dimeric molecular complex maintains its ability to have the biological effector moieties orient in different directions and enable binding to a targeted tissue, cell type, molecule, etc. In one embodiment, the disulfide bond occurs between a cysteine residue in the polypeptide spacer sequence of the first fusion protein and a cysteine residue in the polypeptide spacer sequence of the second fusion protein. In another embodiment, the polypeptide spacer sequences of the first and the second fusion proteins each comprise a hinge region of an IgG, such as an IgG₁, and the disulfide bond attaches cysteine residues from the hinge regions of the first and second fusion proteins to each other. In this embodiment there may be any number of disulfide bonds, such as 2, 3, or 4 disulfide bonds, connecting the IgG hinge regions of the first and the second fusion protein.

### Free Cysteine Residue

The dimeric molecular complexes of the invention are further characterized by having at least one of the fusion proteins with one or more free cysteine amino acid residues capable of conjugating with another molecule. A “free cysteine amino acid residue” refers to a cysteine amino acid residue which is not paired as an intramolecular or intermolecular disulfide bridge. To provide a numerical measure of the ability of a free cysteine amino acid residue to conjugate with another molecule having a functional site reactive to —SH, such free cysteine residues may be characterized as having a thiol reactivity value in the range of 0.6 to 1.0.

The term “thiol reactivity value” is a quantitative characterization of the reactivity of free cysteine amino acid residues. The thiol reactivity value is the percentage of a free cysteine amino acid in a dimeric molecular complex which reacts with a thiol-reactive reagent, and converted to a maximum value of 1. For example, a free cysteine amino acid residue on a dimeric molecular complex which reacts in 100% yield with a thiol-reactive reagent, such as a biotin-maleimide reagent to form a biotin-labelled complex, has a thiol reactivity value of 1.0. Another cysteine amino acid engineered into the same or different fusion protein which reacts in 80% yield with a thiol-reactive reagent has a thiol reactivity value of 0.8. Another cysteine amino acid engineered into the same or different fusion protein that fails totally to react with a thiol-reactive reagent has a thiol reactivity value of 0. Determination of the thiol reactivity value of a particular cysteine may be conducted by ELISA assay, mass spectroscopy, liquid chromatography, autoradiography, or other quantitative analytical tests as are discussed in U.S. Pat. No. 7,521,541.

A free cysteine residue may be created by replacing a non-cysteine residue in a fusion protein with a cysteine residue. A free cysteine residue may also be created by replacing a cysteine residue that is part of a disulfide bond with a non-cysteine residue. Replacement of one cysteine residue in a disulfide bonding pair changes the remaining natural disulfide partner to a free cysteine residue.

The free cysteine residue may occur in any region of the dimeric molecular complex. In one embodiment the free cysteine residue occurs in the biological effector moiety, for example an Fab fragment. As shown in the examples that follow, free cysteine residues were created in the Fab domains or IgE CH4 dimerization domains of dimeric molecular complexes. Free cysteine residues in the Fab dimer or IgE CH4 dimerization domains of the complexes in the examples were generally well tolerated as shown by most of the complexes having binding activity similar to wild type. In another embodiment the free cysteine residue occurs in the polypeptide spacer sequence.

### Conjugates

The conjugates of the invention include a dimeric molecular complex coupled to a conjugate molecule as shown by Formula (I):

D-Lₐ-M,

wherein D is a dimeric molecular complex, L is a linker, M is a conjugate molecule, such as a label, cytotoxic agent, drug, or biocompatible polymer, and a is 0 or 1. When a is 0, then M is covalently attached to D through a free cysteine on D and when a is 1, then L is covalently attached to D through a free cysteine on D.

In one embodiment, the conjugate molecule M may be any molecule that is useful if targeted to a particular tissue, cell type, molecule, or biological location in a mammal. For example, the conjugate molecule may be a label. The term “label” means any moiety which can be attached, preferably covalently, to the linker L or drug M as a conjugate molecule and that functions to: (i) provide a detectable signal (such as a radiolabel or fluorophore); (ii) interact with a second label to modify the detectable signal provided by the first or second label, e.g. FRET (fluorescence resonance energy transfer); (iii) stabilize interactions or increase affinity of binding, with antigen or ligand; (iv) affect mobility, e.g. electrophoretic mobility, or cell-permeability, by charge, hydrophobicity, shape, or other physical parameters, or (v) provide a capture moiety, to modulate ligand affinity, antibody/antigen binding, or ionic complexation.

Suitable labels include biotin, c-myc tag, FLAG tag, fluorescent proteins, horseradish peroxidase, alkaline phosphatase, and β-D-galactosidase. Additional detectable labels are described, for example, in U.S. Pat. No. 4,275,149 and U.S. Pat. No. 4,318,980.

The conjugate molecule may be a “cytotoxic agent,” which refers to a substance that inhibits or prevents the function of cells and/or causes destruction of cells. Cytotoxic agent is intended to include radioactive isotopes (e.g., ²¹¹At, ¹³¹I, ¹²⁵I, ⁹⁰Y, ¹⁸⁶Re, ¹⁸⁸Re, ¹⁵³Sm, ²¹²Bi, ³²P, ⁶⁰C, ³H and radioactive isotopes of Lu), chemotherapeutic agents, and toxins such as small molecule toxins or enzymatically active toxins of bacterial, fungal, plant or animal origin, including synthetic analogs and derivatives thereof. Examples of suitable cytotoxic agents include maytansinoid, Vinca alkaloid, dolastatin, cryptophycin, duocarmycin, calicheamicin, auristatin and anthracyclin. Suitable cytotoxic agents also include bacterial protein toxins Pseudomonas exotoxin A and diphtheria toxin, and the plant protein toxins ricin, abrin, modeccin, saporin, and gelonin. The cytotoxic agent may be a toxophore, for example a polypeptide toxophore or a chemical toxophore. The conjugates of the invention selectively deliver an effective dose of a cytotoxic agent to tumor tissue and thereby are expected to require a lower dosage for efficacy than unconjugated cytotoxic agents.

The conjugate molecule may be a drug. In one embodiment, the drug is an anti-tumor agent that can be targeted to a tumor by use of a biological effector moiety that targets a tumor cell or a molecule involved in tumor growth or cell signaling involved in tumor growth. In another embodiment, the drug is an anti-inflammatory that can be targeted to inflammatory tissue, such as arthritic tissue, using appropriately selected biological effector moieties. Examples of suitable drugs for conjugation include methotrexate and steroids.

Alternatively, the conjugate molecule M may be an entity that provides improved bioavailability to the dimeric molecular complex, such as improved half-life or improved pharmacokinetic profile. Such conjugation would be helpful for example if one or more of the biological effector moieties of the complex has a therapeutically active site and it is desirable to improve bioavailability of the complex. In such embodiments, the free cysteine residue may be conjugated to biocompatible polymers to improve the clearance profile. A biocompatible polymer includes polyalkylene oxides such as without limitation polyethylene glycol (PEG), hydroxyalkyl starches such as without limitation hydroxyethyl starch (HES), dextrans, colominic acids or other carbohydrate based polymers, polymers of amino acids, biotin derivatives, polyvinyl alcohol (PVA), polycarboxylates, polyvinylpyrrolidone, polyethylene-co-maleic acid anhydride, polystyrene-co-malic acid anhydride, polyoxazoline, polyacryloylmorpholine, heparin, albumin, celluloses, hydrolysates of chitosan, starches such as hydroxyethyl-starches and hydroxy propyl-starches, glycogen, agaroses and derivatives thereof, guar gum, pullulan, inulin, xanthan gum, carrageenan, pectin, alginic acid hydrolysates, other bio-polymers and any equivalents thereof. One embodiment involves polyethylene glycol, and another embodiment involves methoxypolyethylene glycol (mPEG). Other useful polyalkylene glycol compounds are polypropylene glycols (PPG), polybutylene glycols (PBG), PEG-glycidyl ethers (Epox-PEG), PEG-oxycarbonylimidazole (CDI-PEG), branched polyethylene glycols, linear polyethylene glycols, forked polyethylene glycols and multi-armed or “super branched” polyethylene glycols (star-PEG).

Methods of conjugation are well known in the art and are described, for example, in Kim et al., 2008, Mol. Cancer. Ther. 7(8): 2486-2497.

The linker L may be absent or may be present. If present, the linker is attached to a free cysteine amino acid residue on the dimeric molecular complex. If the linker is present, it is a bifunctional molecule having at least one functional group reactive with the free cysteine on the dimeric molecular complex. Useful cysteine reactive groups include maleimide, haloacetamide (such as iodoacetamide), vinylsulfone, thiol, triflate, tresylate, aziridine, oxirane, and orthopyridyl disulfate.

The linker thus serves to covalently attach the conjugate molecule to the dimeric molecular complex. It may comprise any molecule that serves as a flexible linker to allow the dimeric molecular complex to orient such that the biological effector moiety is able to target the conjugate to an antigen, substrate, signaling molecule, etc. as appropriate for the effector moiety and that allows for the conjugate molecule to have any necessary distance from the complex and any necessary orientation to allow for therapeutic activity, toxic activity, targeting activity, or activity to improve the bioavailability of the complex. The linker also allows for dimerization of the fusion proteins in the presence of the conjugate molecule. For example, the linker L may be comprised of polypeptides or hydrocarbon chains (including without limitation heteroatoms, heterocycles, aryls, and saturated or unsaturated cyclic carbon groups). Examples of linkers are found in U.S. Pat. No. 7,521,541 and elsewhere in the art.

### Production of Fusion Proteins

Fusion proteins for dimeric molecular complexes of the invention can be produced recombinantly or synthetically, or using a combination of the two approaches. For recombinant production, the invention provides nucleic acid molecules which encode fusion proteins of the invention (see below).

It is possible to produce a fusion protein of the invention using chemical methods to synthesize the amino acid sequence of the fusion protein. Methods include direct peptide synthesis using solid-phase techniques (Merrifield, J. Am. Chem. Soc. 85, 2149-2154, 1963; Roberge et al., Science 269, 202-204, 1995). Protein synthesis can be performed using manual techniques or by automation. Automated synthesis can be achieved, for example, using Applied Biosystems 431 A Peptide Synthesizer (Perkin Elmer). Optionally, fragments of a fusion protein can be separately synthesized and combined using chemical methods to produce a full-length fusion protein. See WO 01/98340.

Nucleic acid molecules of the invention can comprise any nucleotide sequence which encodes the desired fusion protein. Nucleic acid molecules of the invention include single- and double-stranded DNA (including cDNA) and mRNA. Many kits for constructing fusion proteins are available from companies such as Promega Corporation (Madison, Wis.), Stratagene (La Jolla, Calif.), CLONTECH (Mountain View, Calif.), Santa Cruz Biotechnology (Santa Cruz, Calif.), MBL International Corporation (MIC; Watertown, Mass.), and Quantum Biotechnologies (Montreal, Canada; 1-888-DNA-KITS).

Methods which are well known to those skilled in the art can be used to construct nucleic acid molecules of the invention. These methods include in vitro recombinant DNA techniques and synthetic techniques. Such techniques are described, for example, in Sambrook et al. (1989) and in Ausubel et al., Current Protocols in Molecular Biology, John Wiley & Sons, New York, N.Y., 1989.

In some embodiments, the nucleic acid molecules are expression constructs which contain the necessary elements for the transcription and translation of an inserted coding sequence encoding a fusion protein. Fab dimer expression constructs can include a coding sequence for the light chain with a C-terminal cysteine. An expression construct can be present in a vector suitable for introducing fusion proteins of the invention into a cell.

Fusion proteins of the invention can be recombinantly expressed in a variety of host cells. These include, but are not limited to, microorganisms, such as bacteria transformed with recombinant bacteriophage, plasmid, or cosmid DNA expression vectors; yeast transformed with yeast expression vectors, insect cell systems infected with virus expression vectors (e.g., baculovirus), plant cell systems transformed with virus expression vectors (e.g., cauliflower mosaic virus, CaMV; tobacco mosaic virus, TMV) or with bacterial expression vectors (e.g., Ti or pBR322 plasmids), or animal cell systems, particularly mammalian systems, including human systems. See WO 01/98340, which is incorporated herein by reference in its entirety. In one embodiment, the host cells are HKB11 cells. See U.S. Pat. No. 6,136,599. In another embodiment, the host cells are Chinese hamster ovary cells, HEK cells, HeLa cells, 293S cells, or derivatives of one of these cells. The choice of vector components and appropriate host cells is well within the capabilities of those skilled in the art.

In some embodiments of this invention, both fusion proteins of a dimeric molecular complex are expressed in the same cell, preferably from the same plasmid, e.g., as a dicistronic operon (Skerra et al, Protein Eng. 4, 971, 1991). A signal sequence can be included to direct the fusion proteins to the desired cellular location. Expression of the two fusion proteins of a dimeric molecular complex from the same plasmid leads to an increased amount of the bispecific dimer being formed, as equivalent amounts of each component are being produced within the cell.

Optionally, a fusion protein can comprise a moiety which can be used as a detection or purification tag, such as peptides comprising at least five histidine residues, or the commonly used c-myc and FLAG tags.

The dimeric molecular complexes of the invention can be useful for diagnostic purposes. For example, the complex can comprise two fusion proteins, one protein comprising a biological effector moiety designed to bind to an analyte of interest and the other protein comprising a biological effector molecule that is, or is bound to, a detectable label which can easily be quantified, e.g. an enzyme, a fluorescent protein, a radionuclide, etc. In another embodiment one or both biological effector moieties are designed to bind to an analyte of interest and the conjugate molecule is a detectable label.

### Production of Conjugates

Representative methods for conjugation of a cysteine-reactive linker or conjugate molecule to the free cysteine on a fusion protein of the dimer molecular complex are found in U.S. Pat. No. 7,632,921 (especially columns 20-21), Klussman et al. (2004), Bioconjugate Chemistry 15 (4):765-773, and elsewhere in the art. In view of the presence cysteine reactive groups in cell culture medium, for recombinantly produced dimeric molecular complexes it may be necessary to contact the complex with a mild reductant such as Tris(2-carboxyethyl)phosphine (TCEP) to remove the media components that have “capped” the free cysteine and then remove the reductant before reacting the linker with the dimeric molecular complex. See U.S. Pat. No. 7,632,921 and examples infra.

The coupling of the dimeric molecular complex to the linker L or the conjugate molecule M is accomplished by the presence of a reactive moiety such as without limitation maleimide (1), vinylsulfone (2), iodoacetamide (3), or orthopyridyl disulfate (4) on the linker or conjugate molecule.

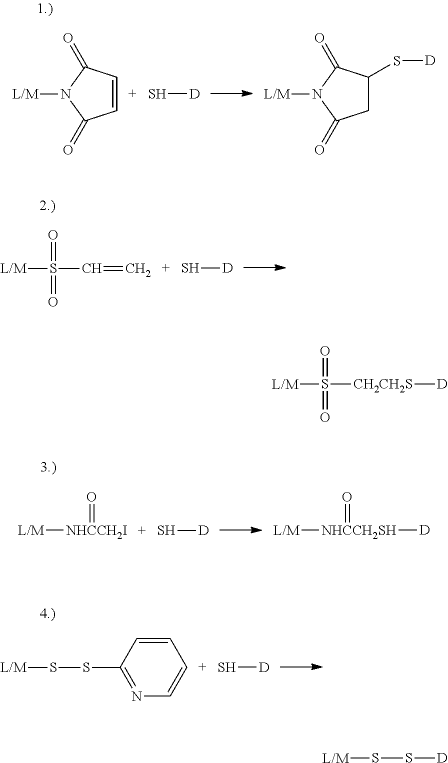

In one embodiment, the dimeric molecular complex is conjugated to a linker or conjugate molecule containing a reactive maleimido group which can covalently bind to the SH-group of the free cysteine. The maleimide group reacts specifically with the free sulfhydryl group provided by the cysteine when the pH of the reaction mixture is between pH 6.5 and 7.5, forming a stable thioether linkage that is not reversible.

### Pharmaceutical Uses, Formulations, Dosages

The conjugates of the invention can be provided in a pharmaceutical composition for administration to a mammal, preferably a human. Conjugates composed of antibody fragments (either mono- or bi-specific) are particularly useful in tumor therapy, such as when a biological effector moiety targets a tumor-associated antigen. For example, the two fusion proteins may comprise biological effector moieties which bind to a tumor marker and the conjugate molecule may comprise a molecule which binds to a T-cell epitope, a cytotoxic agent, or a radionuclide binding peptide or protein to bring a killing function close to the tumor cell.

For preparing suitable pharmaceutical compositions comprising conjugates of the invention, one skilled in the art can use known injectable, physiologically acceptable sterile solutions. For preparing a ready-to-use solution for parenteral injection or infusion, aqueous isotonic solutions, such as, e.g., saline or corresponding plasma protein solutions are readily available. The pharmaceutical compositions may be present as lyophylisates or dry preparations, which can be reconstituted with a known injectable solution directly before use under sterile conditions. A pharmaceutical composition can be supplemented with known carrier substances or/and additives (e.g., serum albumin, dextrose, sodium bisulfite, EDTA, polyol, etc.). Acceptable diluents, carriers, excipients, and stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG).

Pharmaceutical compositions of the invention can be administered by different routes of application known to one skilled in the art, particularly by intravenous injection or direct injection into target tissues. For systemic application, intravenous, intravascular, intramuscular, intraarterial, intraperitoneal, oral, or intrathecal routes can be used. More local administration can be effected subcutaneously, intracutaneously, intracardially, intralobally, intramedullarly, intrapulmonarily or directly in or near the tissue to be treated (connective-, bone-, muscle-, nerve-, epithelial tissue). Depending on the desired duration and effectiveness of the treatment, compositions may be administered once or several times, also intermittently, for instance on a daily basis for several days, weeks or months and in different dosages.

The dosage will depend on age, condition, sex and extent of the disease in the patient and can vary from 0.1 mg/kg to 200 mg/kg, preferably from 0.1 mg/kg or 100 mg/kg/dose, in one or more dose administrations daily, for one to several days. The amount of active ingredient that may be combined with the carrier material to produce a single dosage form will vary depending upon the host treated and the particular mode of administration. If the conjugate is administered by infusion, the conjugate is formulated in order that infusion of a suitable volume at a rate of about 30 mL/hr can occur.

All patents, patent applications, and references cited in this disclosure are expressly incorporated herein by reference in their entireties. The above disclosure generally describes the present invention. A more complete understanding can be obtained by reference to the following specific examples, which are provided for purposes of illustration only and are not intended to limit the scope of the invention.

### EXAMPLES

### Example 1

### Design of Cysteine Mutants of Fab Dimeric Molecular Complexes

This example demonstrates the production of Fab dimeric molecular complexes with the structure shown in FIG. 1 (i.e. a monospecific dimeric molecular complex in which the biological effector moiety is a Fab fragment). The Fab region is derived from the L19 antibody which specifically targets the extra domain B (ED-B) of fibronetin localized to the tumor vasculature (Borsi et al., 2002, Int. J. Cancer 102: 75-85). Dimers of this Fab region are designated as the MN Fab dimer. The Fab regions are covalently attached to a polypeptide spacer sequence consisting of an IgG hinge region. This construct does not have any additional amino acid sequence between the C-terminus of the CH1 domain of the Fab and the N-terminus of the IgG₁ hinge region and the sequence of the CH1 domain and the hinge region is the same as in IgG. The non-mutant form of the Fab dimer is shown in FIG. 1 as pGT821. The amino acid sequence of the light and heavy chains of pGT821 are provided in SEQ ID NO: 1 and SEQ ID NO: 2, respectively. An eight-residue C-terminal sequence from a natural splice variant of IgE called the M2″ extension (WO2007/137760) was added to pGT821 to improve dimer formation and binding activity. This Fab dimer containing the M2″ extension is designated as pGT823. The amino acid sequence of the light and heavy chains of pGT823 are provided in SEQ ID NO: 1 and SEQ ID NO: 3, respectively. The Fab dimer was used with (pGT823) and without (pGT821) the M2″ extension as a framework to introduce free cysteine residues by three different approaches.

First, one or the other of the existing cysteine groups involved in the disulfide bonds connecting the light and heavy chains of pGT821 and 823 have been removed to leave a free cysteine group on either the C-terminus of the CL domain of the light chain or the CH1 domain of the heavy chain (FIG. 1).

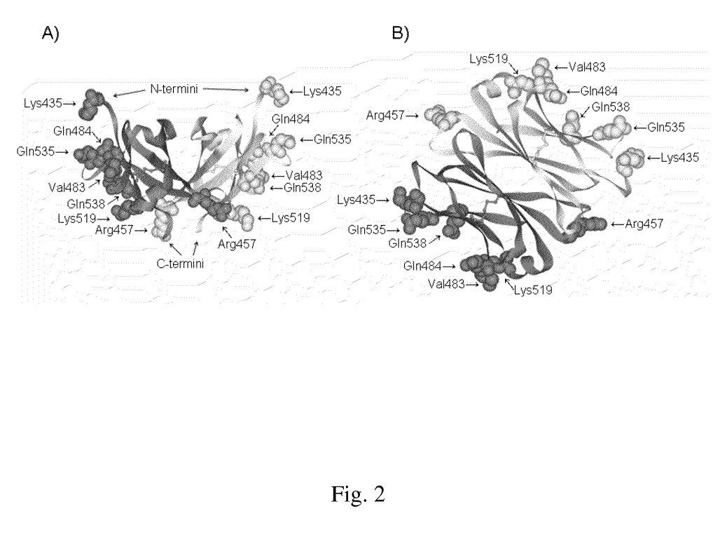

In a second design approach to create unique free cysteine residues, the solvent exposed surface was calculated for all of the side chains on the IgE CH4 domain present in the Fab dimer using the program Insight II (Accelrys, Inc., San Diego, Calif., USA). For residues with at least 90 Å² or more of solvent exposure in either one or both dimer domains, the stability after substitution with cysteine was calculated using two different algorithms for both chemical and thermal stability using the program CUPSAT (Parthiban et al., 2006, Nucleic Acids Res. 34 (Web Server Issue): W239-242). These results suggested a number of positions in the IgE CH4 domain that were good candidates for mutation to cysteine. These positions were predicted to be stable after mutation to cysteine in either one or both dimer domains by at least one or more of the algorithms (chemical or thermal), and include: Thr434, Lys435, Ser437, Pro439, Arg440, Pro451, Glu452, Trp453, Pro454, Arg457, Asp458, Lys459, Asp473, Glu482, Val483, Gln484, Asp487, Ala488, Gln494, Arg496, Lys497, Lys499, Ser501, Arg513, Glu517, Gln518, Lys519, Ser532, Pro533, Ser534, Gln535, Gln538, Arg539, Asn544, Pro545 and Gly546, with numbering based on their position in the full-length IgE protein. Further, by visual inspection of these positions, seven suitable positions were chosen: Lys435, Arg457, Val483, Gln484, Gln535, Gln 538, and Lys519. After further inspection, three finalists were chosen based on several considerations: the new cysteine should not be to be too close to existing disulfides in the hinge, in the immunoglobulin domain itself (Cys464 and Cys524), or in the M2″ extension; the new cysteine must not be able to form a disulfide with itself; and the new cysteine must be solvent exposed and available for conjugation. Based on these considerations the first residues in the IgE CH4 mutated to cysteine were Gln538, Lys519, and Val483 (FIG. 2). Finally, following the results with these first three mutations, a third design approach was tested based upon the structure of the IgE CH4 domain. A second set of mutations were made in the CH4 domain region in which four alanine and two serine residues were changed to cysteine: Ala441, Ala488, Ala514, Ser532, Ala540 and Ser542.

### Example 2

### Plasmid Construction

Mutations were introduced into DNA encoding the MN Fab dimer by the QuickChange mutagenesis kit (Stratagene, Agilent Technologies Inc., Santa Clara, Calif.). Oligonucleotides used to introduce the mutations were as follows:

Fab CH1 C243:
(SEQ ID NO: 68)
ggataaaaaagtggaaccgaagagctccgataaaactcacac
Fab CL C234:
(SEQ ID NO: 69)
caggggagagtcttaagcggccgcgcc
IgE CH4 V483C
(SEQ ID NO: 70)
gctacacaacgagtgccagctgccggacgcccgg
IgE CH4 K519C
(SEQ ID NO: 71)
gggccgaatgggagcagtgcgatgaattcatctgccgtgc
IgE CH4 Q538C
(SEQ ID NO: 72)
cccctcacagaccgtctgcagagcggtgtctgtaaatccc
IgE CH4 A441C
(SEQ ID NO: 73)
gaccagcggcccacggtgtgccccggaagtc
IgE CH4 A488C
(SEQ ID NO: 74)
gcagctcccggactgcagacacagcacgacgcagc
IgE CH4 A514C
(SEQ ID NO: 75)
gccgcctggaggtcaccaggtgcgaatgggagc
IgE CH4 S532C
(SEQ ID NO: 76)
gtccatgaggcagcatgcccctcacagacc
IgE CH4 A540C
(SEQ ID NO: 77)
cctcacagaccgtccagcgctgcgtgtctgtaaatcccg
IgE CH4 S542C
(SEQ ID NO: 78)
ccagcgagcggtgtgtgttaaccccgagtcatcg

Generation of free cysteines by mutation of existing cysteines in the disulfide bonds connecting the light and heavy chain of the MN Fab dimer.

Since the MN Fab dimer expression plasmids for pGT821 (−M2″) and pGT823 (+M2″) are very large, it was necessary to construct smaller plasmids suitable for mutagenesis. Therefore, large fragments from pGT821 and pGT823 containing both the light and heavy chain genes were transferred to pBluescript to make pGT833 and pGT834, respectively. Mutagenesis was performed on pGT833 and pGT834 with the QuickChange kit (Stratagene, Agilent Technologies Inc., Santa Clara, Calif.). Mutant clones were verified by sequencing and the mutations were transferred back to the expression vectors on smaller subfragments.

Insertion of Free Cys Residues into the IgE CH4 Region.

The three solvent-exposed residues in the IgE CH4 region described above (V483, K519, and Q538) were mutated to cysteine and transferred back to the parental expression vectors in the same way as the cysteine to serine mutations. Table 1 contains a summary of all the plasmids used in this study.

TABLE 1
Expression plasmids for Fab dimers containing free cysteine
residues. Residues in the Fab region (CL C234 and CH1 C243)
are numbered from the N-terminal of the light and heavy chains
of the Fab dimer. Residues in the IgE CH4 region are numbered
from the N-terminal of the natural IgE heavy chain.

| plasmid | free Cys | M2″ |
|---|---|---|
| pGT821 | none | − |
| pGT823 | none | + |
| pGT841 | Fab CL C234 | − |
| pGT842 | Fab CL C234 | + |
| pGT843 | Fab CH1 C243 | − |
| pGT844 | Fab CH1 C243 | + |
| pGT853 | IgE CH4 K519C | − |
| pGT854 | IgE CH4 K519C | + |
| pGT855 | IgE CH4 Q538C | − |
| pGT856 | IgE CH4 Q538C | + |
| pGT857 | IgE CH4 V483C | − |
| pGT858 | IgE CH4 V483C | + |
| pGT2008 | IgE CH4 A441C | + |
| pGT2009 | IgE CH4 A488C | + |
| pGT2010 | IgE CH4 A514C | + |
| pGT2011 | IgE CH4 S532C | + |
| pGT2012 | IgE CH4 A540C | + |
| pGT2013 | IgE CH4 S542C | + |

### Example 3

### Protein Expression, Purification and Characterization

Human HKB11 suspension cells (22.5 ml at 2.2 E6 cells/ml in 293 FreeStyle media) were transfected with 50 μg of plasmid DNA in 4.0 ml OptiMEM containing 70 μl 293Fectin (Invitrogen, Carlsbad, Calif.). After 3 hrs at 37° C., 25 ml of RF283 media containing 2% Ultra-low IgG FBS was added and the transfected cells were shaken at 37° C. for 3 days. Cells were pelleted, the media collected, and the cells were resuspended in 50 ml RF283 media containing 1% Ultra-low IgG FBS. After 3 days shaking at 37° C., the cells were pelleted and the media pooled with the first collection. Conditioned media was concentrated to 10 ml with Amicon concentrators and MN Fab dimer protein was purified on 5-ml Protein L columns (Pierce) as per the vendor's instructions. The protein concentration was measured by absorbance at 280 nm.

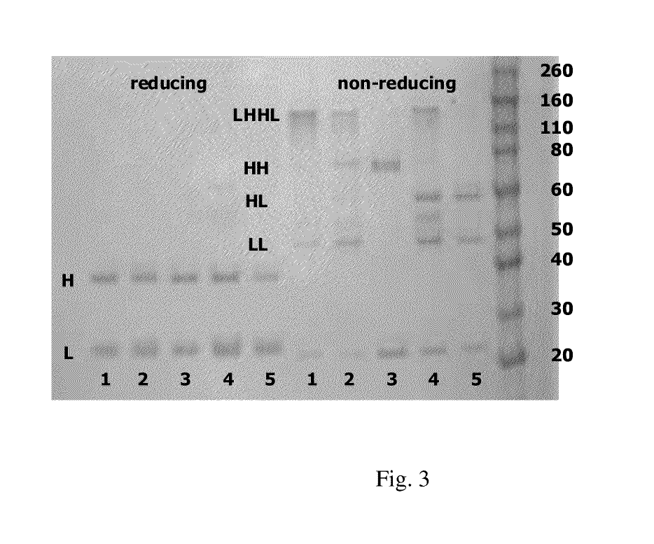

Purified proteins were separated by reducing or non-reducing SDS-PAGE, and visualized by FastBlue staining (FIG. 3). All proteins appeared similar under reducing conditions. The H and L chains of pGT842 and 844 dissociated under non-reducing conditions. pGT854 and 856 had less complete Fab dimer and more HL monomer than the WT Fab dimer, suggesting that dimerization is somewhat impaired by mutations in the IgE CH4 domain. The proportion of dimer species was much less in the muteins lacking the M2″ extension (data not shown), so these muteins were not pursued.

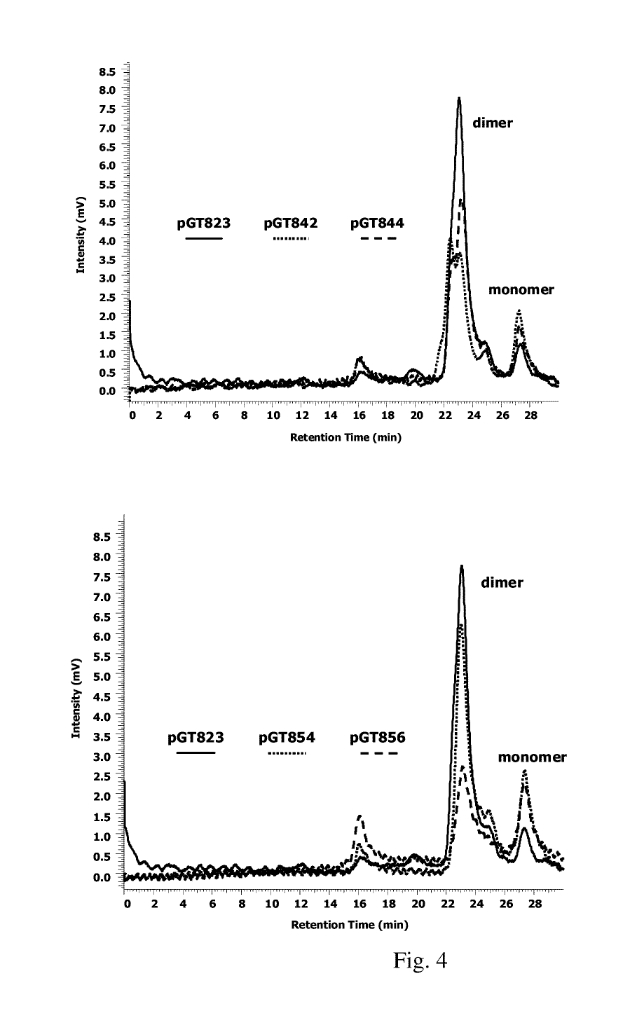

Proteins were separated by size-exclusion chromatography (SEC) on silica-based TSK gel G3000 SW columns (Tosoh Biosciences) in 200 mM KH₂PO₄/150 mM KCl pH 6.8, and peaks were quantified (FIG. 4). The proportion of dimer and monomer species (Table 2) correlated well with the appearance on the SDS gel.

TABLE 2
Proportion of dimer and monomer species
in Fab dimer free cysteine variants.

| plasmid | % dimer | % monomer |
|---|---|---|
| pGT823 | 93 | 7 |
| pGT842 | 82 | 18 |
| pGT844 | 88 | 12 |
| pGT854 | 81 | 19 |
| pGT856 | 62 | 38 |
| pGT2008 | 55 | 45 |
| pGT2009 | 82 | 18 |
| pGT2010 | 67 | 33 |
| pGT2011 | 55 | 45 |
| pGT2012 | 49 | 51 |
| pGT2013 | 44 | 56 |

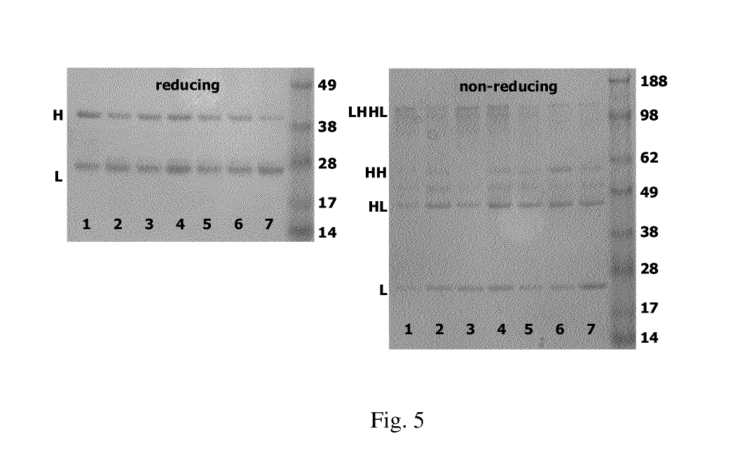

The second set of alanine and serine residues in the IgE CH4 region (Ala514, Ser532, Ala540 and Ser542) was expressed in human cells and purified on Protein L columns. Purified proteins were examined by SDS-PAGE (FIG. 5). Dimer formation was reduced to various extents by these mutations.

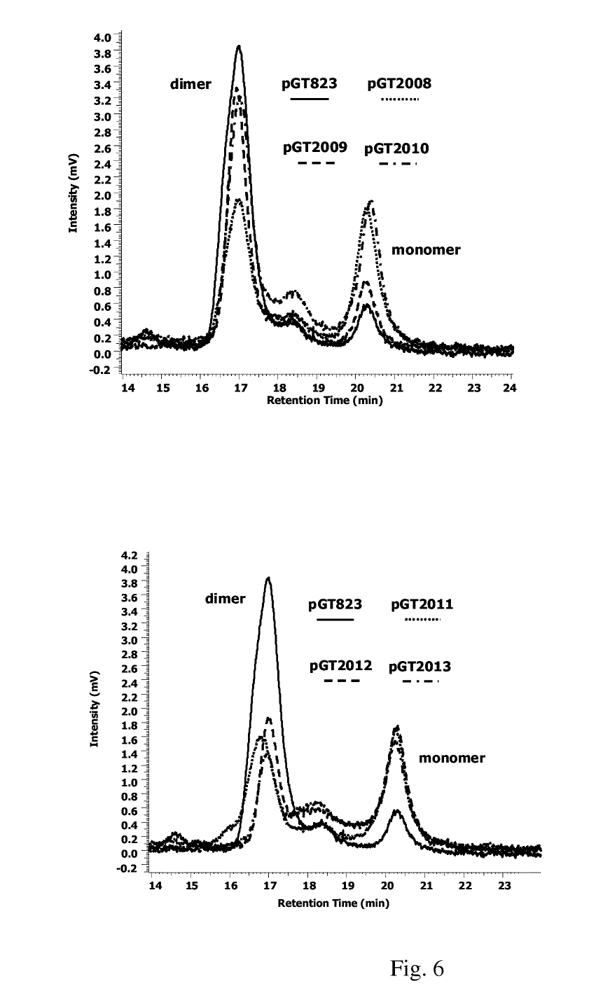

This series of IgE CH4 free cysteine muteins was then examined by SEC (FIG. 6). The relative amounts of dimer and monomer species (Table 2) correlated fairly well with the appearance on the SDS gel.

### Example 4

### Functional Binding by ELISA

The binding activity of the proteins on immobilized MN antigen was assayed by ELISA. MN antigen was coated on Immulon HBX plates in PBS overnight. Antigen was removed and the plates were blocked with PBS/2% BSA. Protein dilutions in PBS/1% BSA were bound for 2 hr at room temperature. Samples were removed, the plate was washed with PBS/0.05% Tween 20, and HRP-conjugated anti-Fab or anti-IgE antibody was added. After 30-60 min the detection antibody was removed and the plate washed. Detection reagent Amplex Red was added and the signal quantified with a fluorescent plate reader.

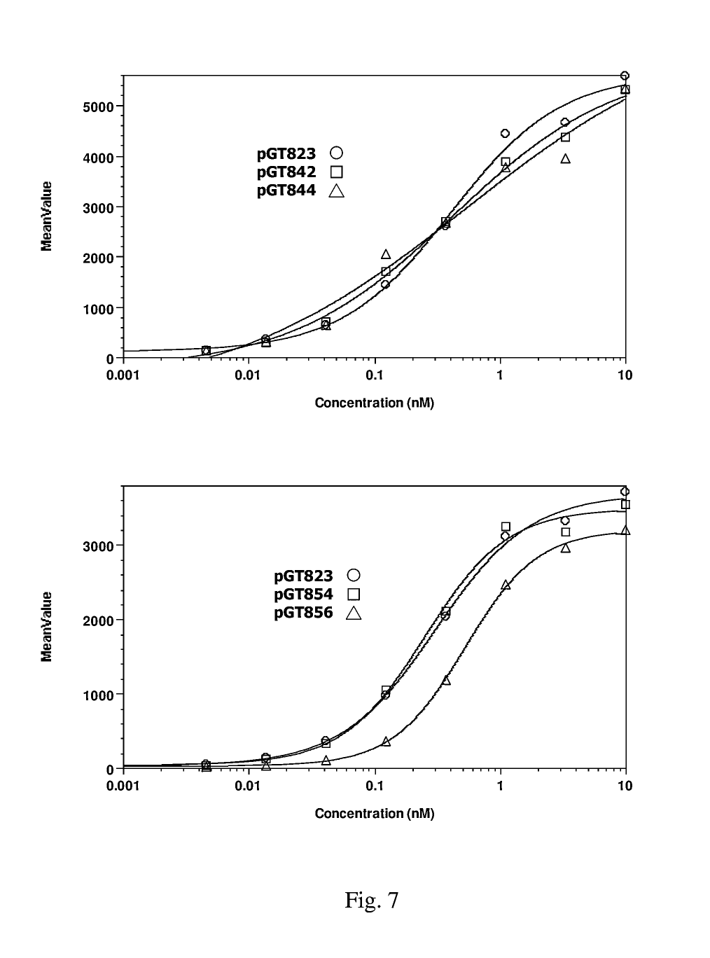

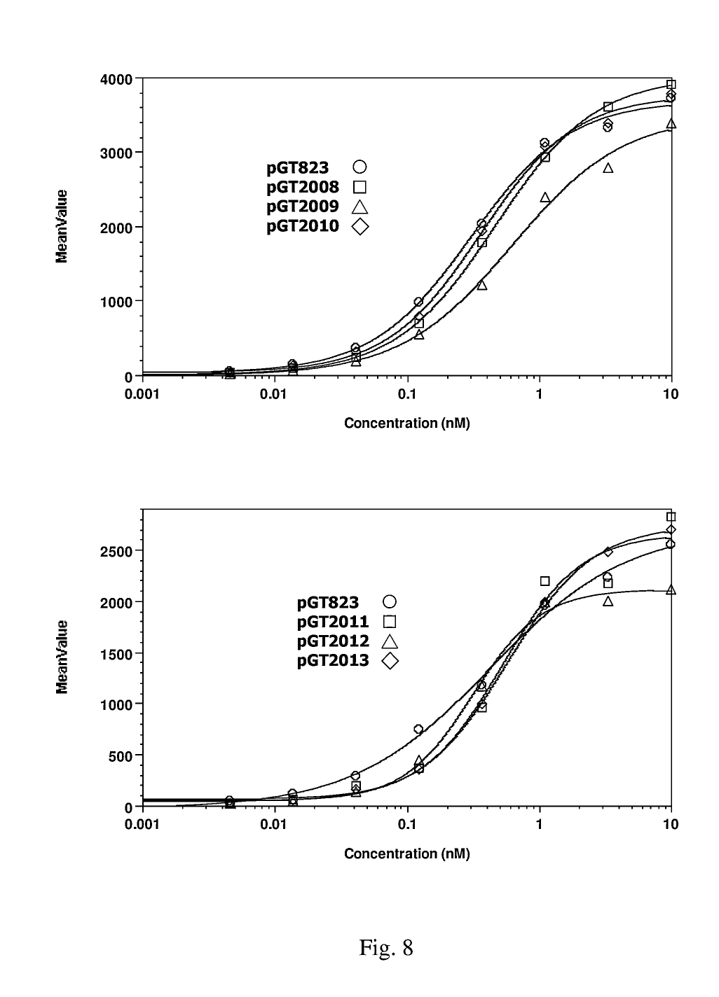

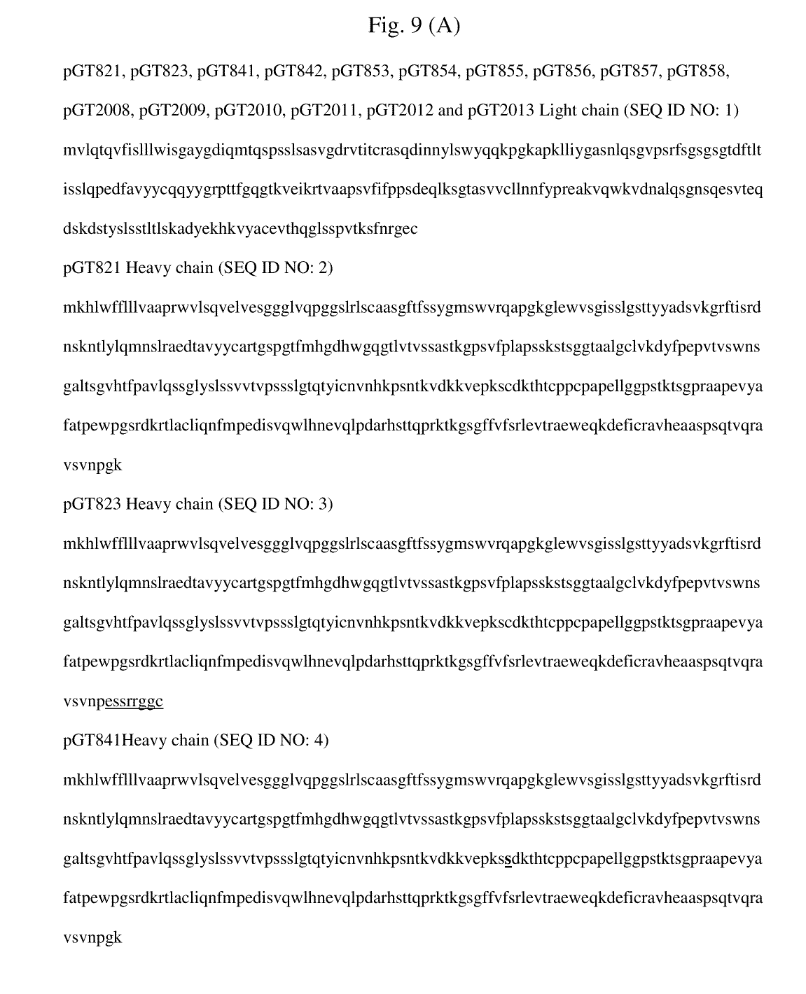

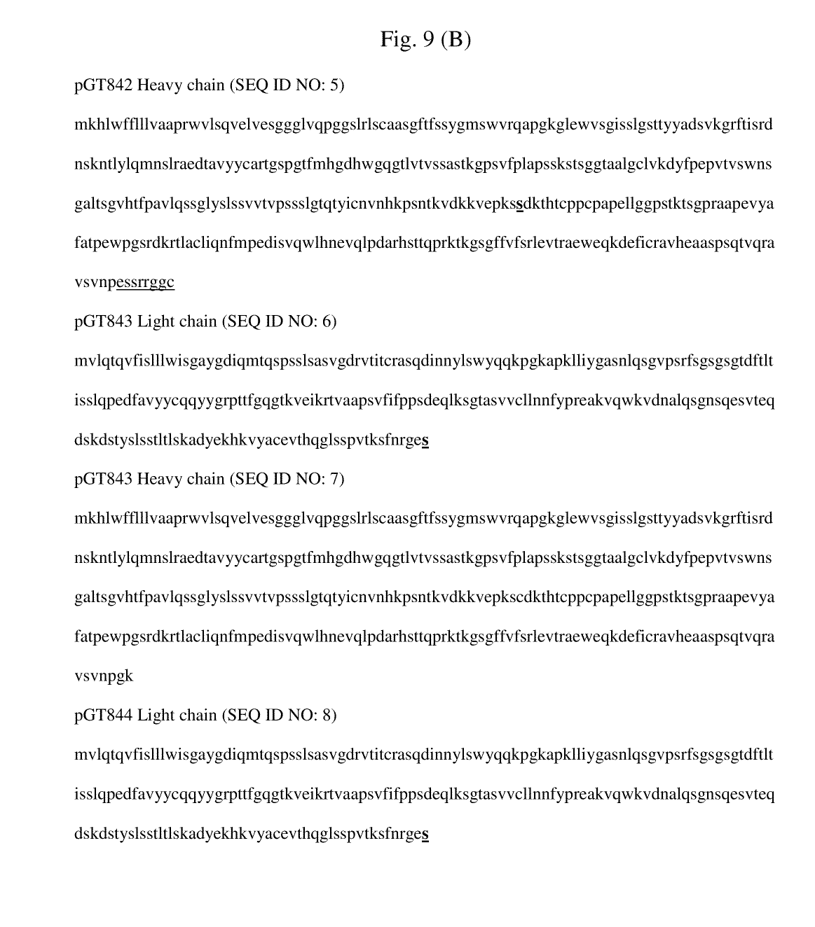

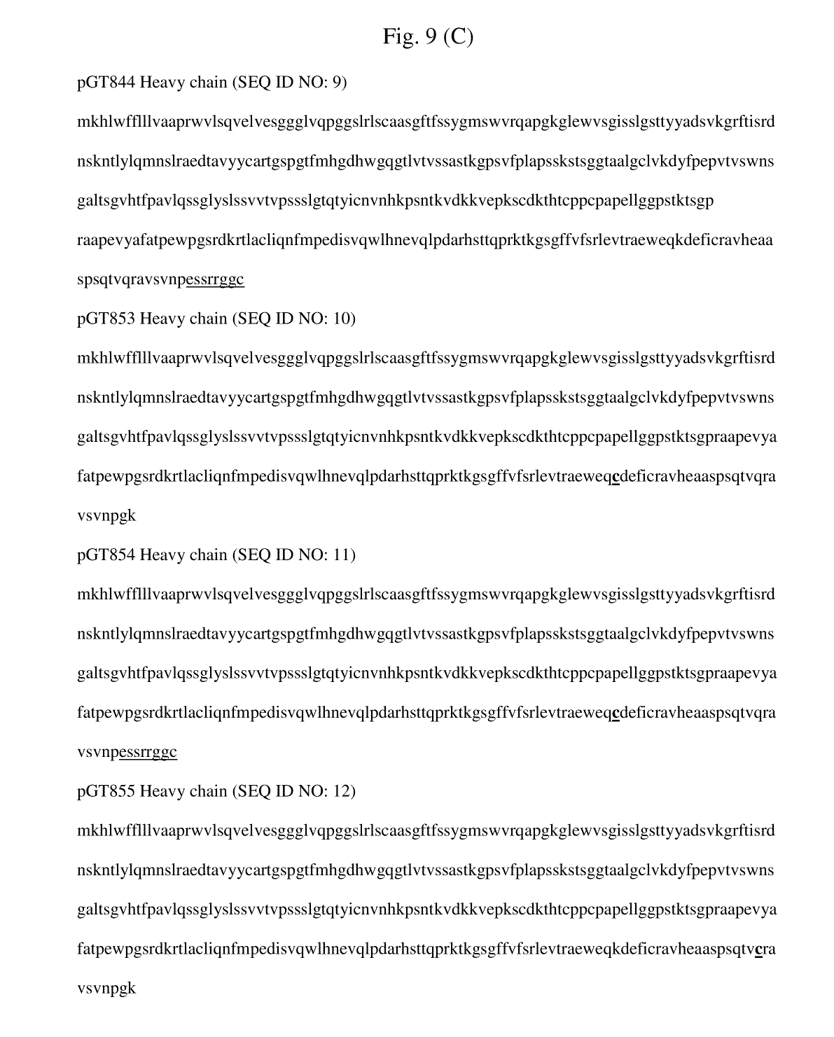

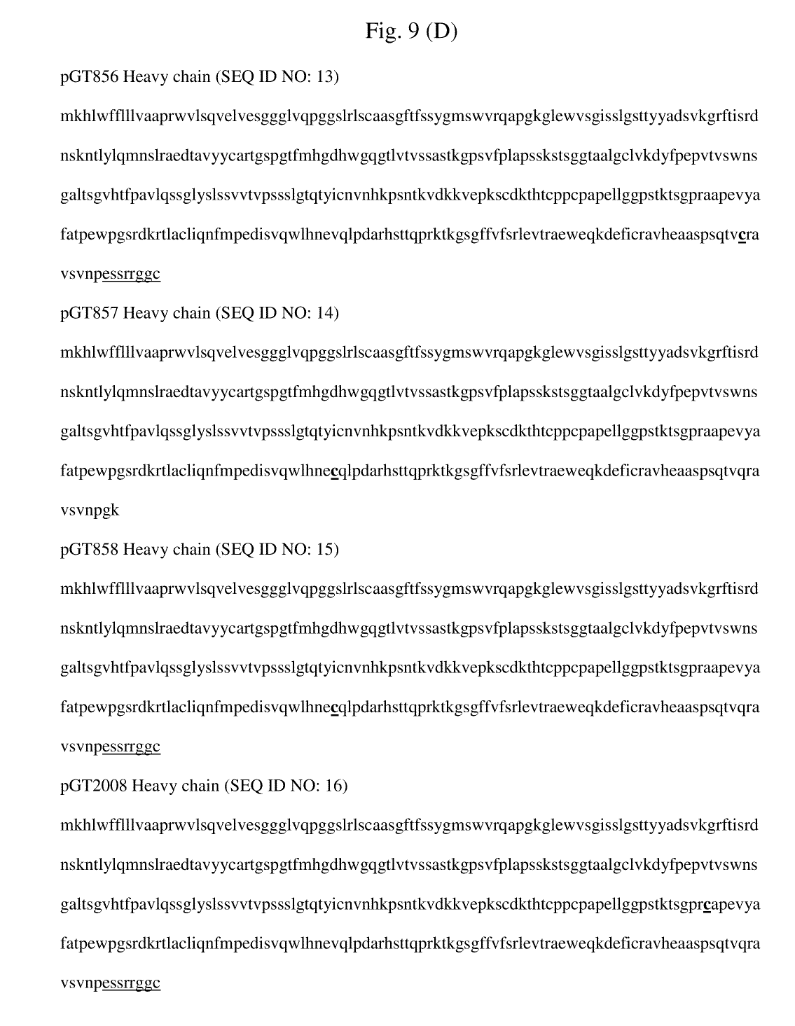

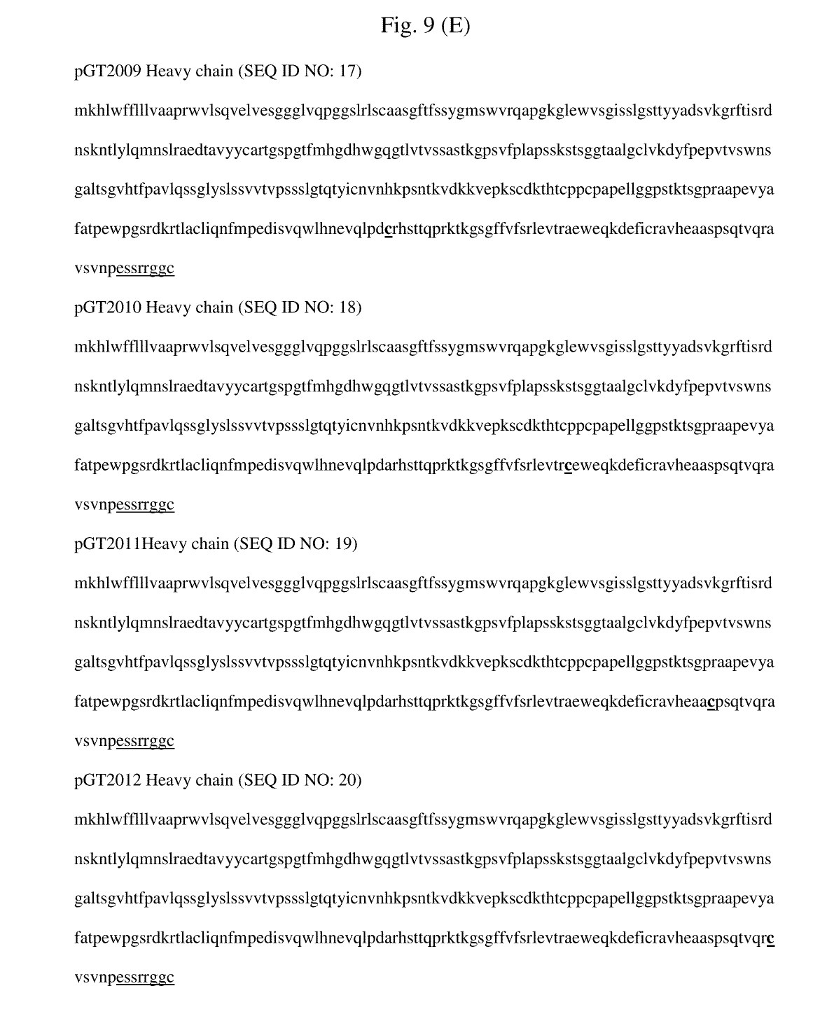

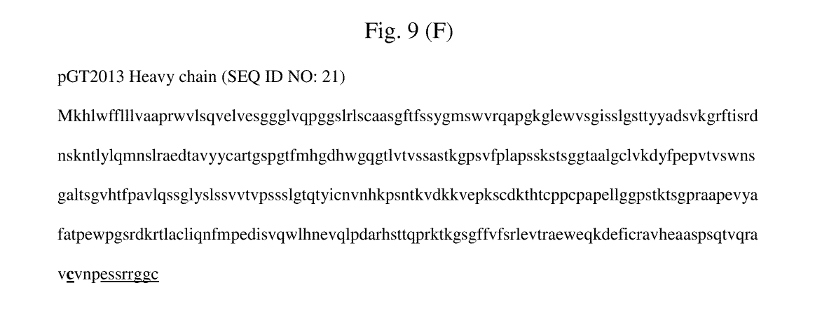

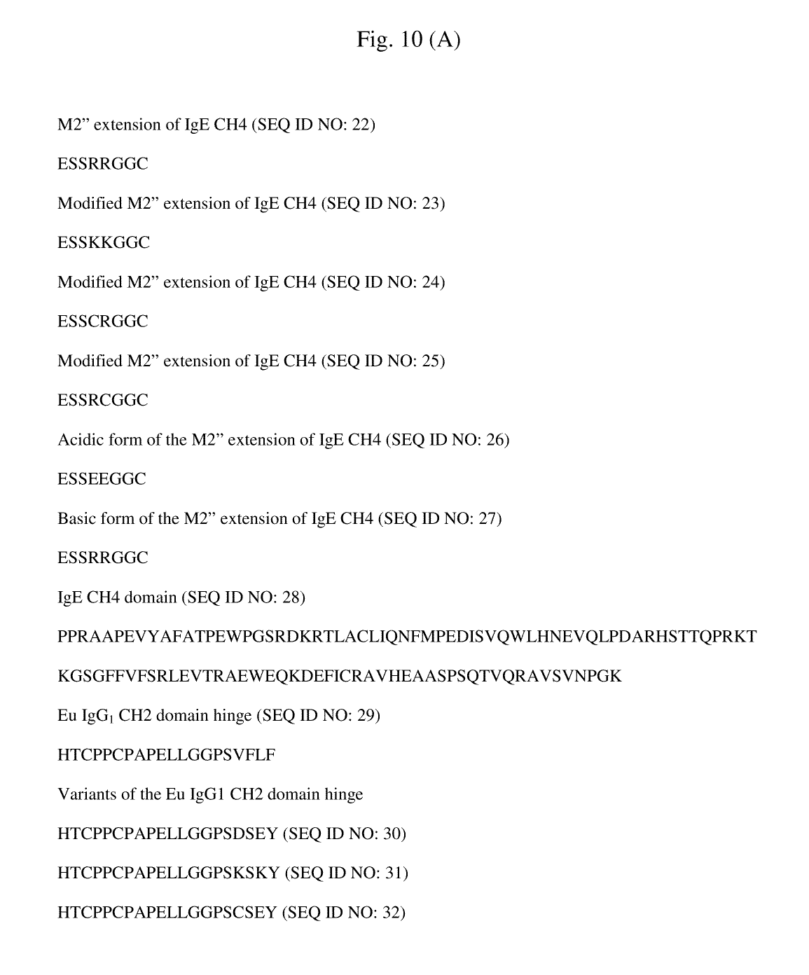

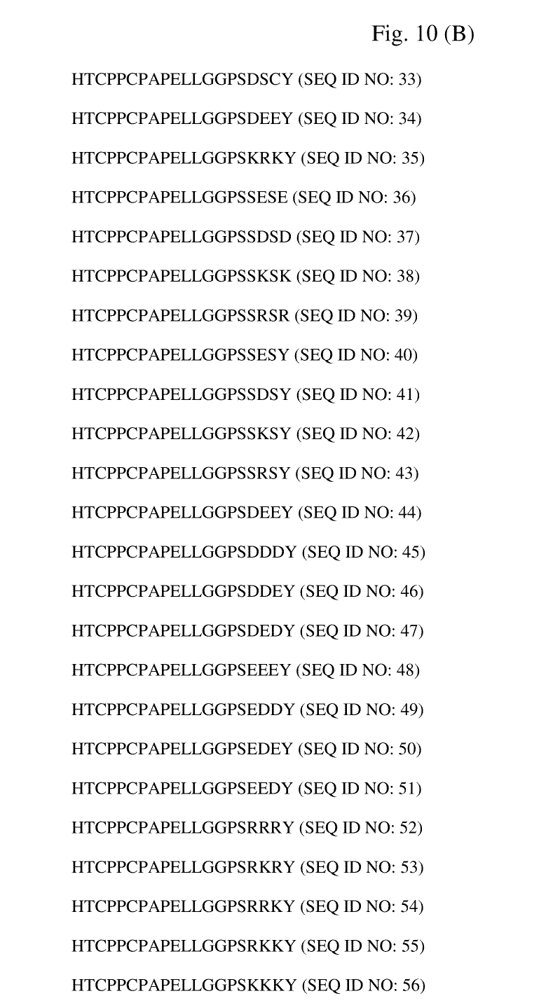

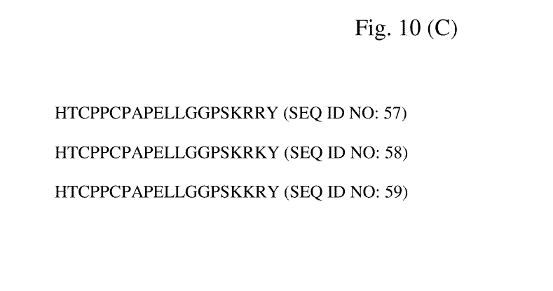

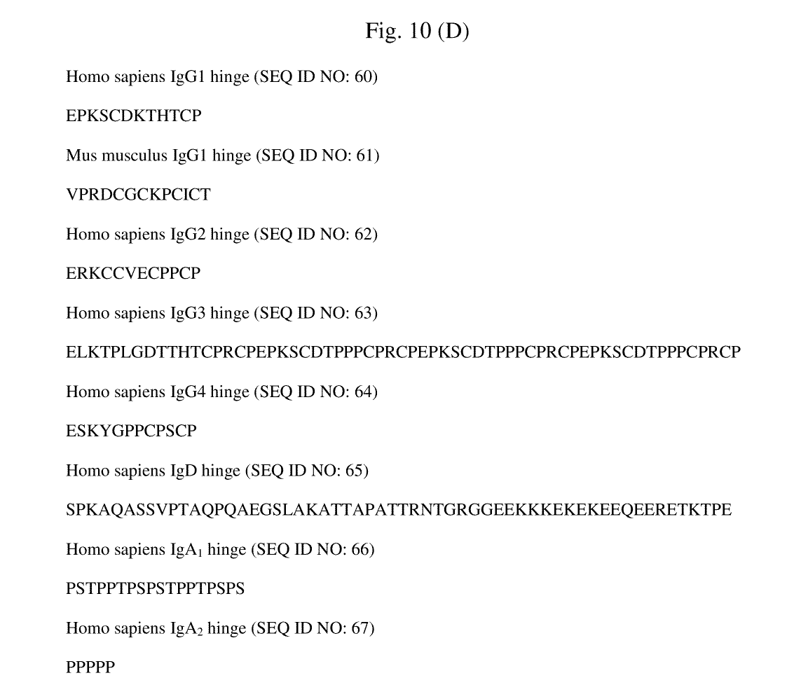

The binding activity of the pGT842, 844, and 854 muteins was about the same as the WT, whereas the binding activity of pGT856 was somewhat lower (FIG. 7). The second set of cysteine mutants (Ala514, Ser532, Ala540 and Ser542) was also found to have functional antigen binding, as shown in FIG. 8.

### Example 5

### Conjugation of Free Cysteines

The free cysteine in each mutant may be conjugated directly after purification of the antibody format or following limited reduction to remove mixed disulfides. It is well known that free cysteines may form mixed disulfides with low molecular weight thiol containing compounds found in cells and biological media such as cysteine or glutathione (Glaser et al., 1982, Int. J. Pept. Protein Res. 20: 56-62; Summa et al., 2007, Proteins 69: 369-378; Manukyan et al., 2008, Thrombosis Res. 122: S19-S22). If present, these mixed disulfides are first reduced using mild reduction conditions based on modifications of published protocols (Glennie et al., 1987, J. Immunol. 139: 2367-2375; Keler et al., 1997, Cancer Res. 57: 4008-4014). An aqueous solution of the purified antibody format protein is first reduced in 0.1 to 100 mM mercaptoethanol or mercaptoethanolamine, preferably 10 mM ethanolamine, for 10 to 300 minutes, preferably 30 minutes, at 10 to 50° C., preferably 30° C. The protein is then quickly chilled to 4° C. and separated from the low molecular weight thiol by either dialysis or SEC at low pH to minimize oxidation of the free cysteine in the protein, preferably SEC on Sephadex 0-25 equilibrated in 50 mM sodium acetate, 0.5 mM EDTA, pH 5.3. The antibody or antibody fragment may also be reduced by incubation with 5 mmol/L Tris (2-carboxyethyl)-phosphine (TCEP) at 37° C. for 1.5 h. Excess TCEP can be removed by dialysis. The antibody format protein is collected and mixed with the activated linker or activated linker-conjugate molecule and conjugated to the dimeric molecular complex.

In one embodiment, the dimeric molecular complex may be conjugated via its engineered free cysteine residue(s) to maleimidocaproyl-valine-citrulline-p-aminobenzyloxycarbonyl-monomethylauristatin E (mc-vcMMAE) or maleimidocaproyl-valine-citrulline-p-aminobenzyloxycarbonyl monomethylauristatin F (mc-vcMMAF) (Kim et al., 2008, Mol. Cancer. Ther. 7(8): 2486-2497). The mc-vcMMAE or mc-vcMMAF is added in slight excess to maximize binding to free cysteine residues. The identities of the resultant conjugates can be confirmed by liquid chromatography-mass spectrometry using a PLRP-S reverse-phase column (Polymer Labs) coupled to a QTOF-API high-resolution mass spectrometer (Waters). Drug conjugates are immediately frozen and stored at −80° C. until use.

### Example 6

### In Vivo Biodistribution of the Conjugate

In vivo biodistribution of the conjugate is determined by radiolabeling of the complex, injection of the labeled conjugate into mice, and analysis of the radioactivity of different tissue types. For example, targeting of tumor tissue may be determined by injection and analysis of tumor bearing mice. Methods for radiolabeling and chelator attachment are adapted from Nikula et al. (1995), Nucl. Med. Biol., 22, 387-390. Purified conjugate is characterized by SEC and SDS-PAGE prior to use. Labeling is performed using ¹¹¹In, in buffers and equipment which is rendered metal-free by repeated rinsing with 10 mM EDTA solution and Chelex-treatment prior to filtration. The chelator p-SCN-CHX-A″-DPTA may be obtained from Macrocyclics Inc. (Dallas, Tex.). The buffer used for conjugation contains 50 mM carbonate, 150 mM NaCl, pH 6.5. Radiolabeling is performed in a buffer containing 50 mM NaAc, 150 mM NaCl, pH 6.5.

Solutions of ¹¹¹In-radiolabeled conjugate in PBS buffer are injected into eight LNCaP tumor bearing mice (2/vCi per animal). Two animals are weighed and tissues from the animals are harvested at 15 min, 3 hr, 6 hr, and 48 hr following injection of labeled antibody. The tumor, tissue, or blood sample is weighed and the total radioactivity determined. The mCi per gram of sample is compared with the total mCi per animal weight to determine the percentage of the injected dose per gram tissue and the average calculated for each group and time point (mean % ID per gram).

### Example 7

### Large Scale Production of Dimeric Molecular Complex Conjugates

Addition of free cysteine residues to the IgE CH4 domain was generally well-tolerated by the molecule, with most of the muteins having binding activity similar to the WT, despite appearing to have proportionally less dimer and more partial species by SDS-PAGE and SEC. These muteins are tested for accessibility to conjugation in the absence of prior chemical reduction. The muteins are produced by large-scale expression and physical characterization of muteins is performed by SDS-PAGE, Western blot, and SEC. The binding activity of the muteins in vitro is determined by ELISA and Biacore analysis, and their activity on cells by binding and internalization assays in vitro. In vivo assays are used to examine tumor localization, biodistribution, and clearance from circulation. Following conjugation to the toxophore, the conjugated products are examined by SEC-MALS and RP-HPLC to determine drug load. The binding affinity of the conjugated proteins is determined, and the cytotoxic effect on tumor cells in culture is assayed. In vivo assays include tumor localization, biodistribution, clearance, toxicity, and efficacy on tumor-bearing mice. Free cysteine residues in the IgE CH4 domain that are effectively conjugated in the MN Fab dimer are introduced into other dimeric molecular complexes with different biological effector moieties, allowing the development of a broad platform for dimeric molecular complex conjugates.

1. A dimeric molecular complex comprising a first and a second fusion protein, wherein each fusion protein comprises from its N to C terminus:
(A) a biological effector moiety selected from the group consisting of:
(1) a single chain antibody;
(2) an Fab fragment;
(3) an extracellular domain of a type I membrane receptor;
(4) a cytokine;
(5) a chemokine;
(6) an enzyme; and
(7) a toxin; wherein at least one of the fusion proteins has a biological effector moiety that is a targeting biological effector moiety selected from the group consisting of (1) a single chain antibody; (2) an Fab fragment; (3) an extracellular domain of a type I membrane receptor; (4) a cytokine; (5) a chemokine; and (6) an enzyme;
(B) a polypeptide spacer sequence; and
(C) a CH4 dimerization domain of an IgE molecule covalently bound to the polypeptide spacer sequence;
wherein the molecular complex comprises at least one disulfide bond between a cysteine residue in the first fusion protein and a cysteine residue in the second fusion protein, and at least one of the fusion proteins comprises one or more free cysteine amino acid residues.

2. The molecular complex of claim 1, wherein the biological effector moieties of the first and second fusion proteins each comprises an antigen binding site.

3. (canceled)

4. (canceled)

5. The molecular complex of claim 1, wherein i) cysteine residue in the biological effector moiety is replaced with a non-cysteine residue to create a free cysteine residue, or ii) a non-cysteine residue in the CH4 dimerization domain is replaced with a cysteine residue to create a free cysteine residue.

6. (canceled)

7. The molecular complex of claim 1, wherein i) the CH4 dimerization domain comprises the amino acid sequence of SEQ ID NO: 28, or ii) the CH4 dimerization domain comprises an amino acid sequence of SEQ ID NO: 28 that is mutated such that a non-cysteine residue at position Ala4, Val146, Ala51, Ala77, Lys82, Ser95, Gln101, Ala103 or Ser105 is replaced with a cysteine residue.

8. (canceled)

9. The molecular complex of claim 1, wherein the first and the second fusion protein each further comprises an M2″ extension of an IgE splice variant located between the CH4 dimerization domain and the C-terminus of the protein.

10. The molecular complex of claim 1, wherein the polypeptide spacer sequences of both the first and second fusion protein each comprises an IgG hinge.

11. (canceled)

12. The molecular complex of claim 10, wherein the hinge region of at least one of the first and the second fusion protein comprises an amino acid sequence selected from the group consisting of SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, and SEQ ID NO: 59.

13. The molecular complex of claim 1, wherein the polypeptide spacer sequences of both the first and the second fusion proteins each comprises 10-500 amino acids, and wherein each of the polypeptide spacer sequences is covalently bound to the biological effector moiety and the CH4 dimerization domain.

14. A dimeric molecular complex comprising a first and a second fusion protein, wherein each fusion protein comprises from its N to C terminus:
(A) an IgE CH4 dimerization domain with a C terminal extension comprised of an M2″ extension of an IgE CH4 splice variant;
(B) an amino acid spacer which is covalently bound to the IgE CH4 dimerization domain; and
(C) an extracellular domain of a type II membrane receptor;
wherein the molecular complex comprises at least one disulfide bond between a cysteine residue in the first fusion protein and a cysteine residue in the second fusion protein, and at least one of the fusion proteins comprises one or more free cysteine amino acid residues.

15. A nucleic acid molecule which encodes a fusion protein comprising from N to C terminus:
(A) a biological effector moiety selected from the group consisting of:
(1) a single chain antibody;
(2) an Fab fragment;
(3) an extracellular domain of a type I membrane receptor;
(4) a cytokine;
(5) a chemokine;
(6) an enzyme; and
(7) a toxin;
(B) a polypeptide spacer sequence; and
(C) a CH4 dimerization domain of an IgE molecule covalently bound to the polypeptide spacer sequence;
wherein the fusion protein comprises one or more free cysteine amino acid residues.

16. The nucleic acid molecule of claim 15, wherein the biological effector moiety comprises an antigen binding site.

17. The nucleic acid molecule of claim 15, wherein a non-cysteine residue in the CH4 dimerization domain is replaced with a cysteine residue to create the free cysteine residue.

18. The nucleic acid molecule of claim 15, wherein i) the CH4 dimerization domain comprises the amino acid sequence of SEQ ID NO: 28, or ii) the CH4 dimerization domain comprises an amino acid sequence of SEQ ID NO: 28 that is mutated such that a non-cysteine residue at position Ala4, Val46, Ala51, Ala77, Lys82, Ser95, Gln101, Ala103 or Ser105 is replaced with a cysteine residue.

19. (canceled)

20. The nucleic acid molecule of claim 15, wherein the fusion protein further comprises an M2″ extension of an IgE splice variant located between the CH4 dimerization domain and the C-terminus of the protein.

21. The nucleic acid molecule of claim 15, wherein the polypeptide spacer sequence comprises an IgG hinge.

22. (canceled)

23. The nucleic acid molecule of claim 21, wherein the hinge region comprises an amino acid sequence selected from the group consisting of SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, and SEQ ID NO: 59.

24. The nucleic acid molecule of claim 15, wherein the polypeptide spacer sequence comprises 1-200 amino acids, and is covalently bound to the biological effector moiety and the CH4 dimerization domain.

25. An expression cassette comprising the nucleic acid molecule of claim 15.

26. A host cell comprising the nucleic acid molecule of claim 15.

27. A conjugate comprising a dimeric molecular complex coupled to a drug moiety, cytotoxic agent, label, or biocompatible polymer as shown in (I):
D-Lₐ-M (I)
wherein D is the dimeric molecular complex, L is a linker covalently attached to D at a free cysteine on D, M is a conjugate molecule selected from a drug moiety, cytotoxic agent, label, or biocompatible polymer, and a is 0 or 1 and further wherein the dimeric molecular complex comprises a first and a second fusion protein, wherein each fusion protein comprises from its N to C terminus:
(A) a biological effector moiety selected from the group consisting of:
(1) a single chain antibody;
(2) an Fab fragment;
(3) an extracellular domain of a type I membrane receptor;
(4) a cytokine;
(5) a chemokine;
(6) an enzyme; and
(7) a toxin; wherein at least one of the fusion proteins has a biological effector moiety that is a targeting biological effector moiety selected from the group consisting of (1) a single chain antibody; (2) an Fab fragment; (3) an extracellular domain of a type I membrane receptor; and (4) an enzyme;
(B) a polypeptide spacer sequence; and
(C) a CH4 dimerization domain of an IgE molecule covalently bound to the polypeptide spacer sequence;
wherein the molecular complex comprises at least one disulfide bond between a cysteine residue in the first fusion protein and a cysteine residue in the second fusion protein, and at least one of the fusion proteins comprises one or more free cysteine amino acid residues that reacts with the linker to form the conjugate or, if a is 0, reacts with the conjugate molecule to form the conjugate.

28. The conjugate of claim 27, wherein at least one of the biological effector moieties of the first and second fusion proteins comprises an antigen binding site.

29. (canceled)

30. (canceled)

31. The conjugate of claim 27, wherein M is a cytotoxic agent and the biological effector moiety comprises an antigen binding site for a cancerous cell.

32. The conjugate of claim 27, wherein M is a drug moiety and the biological effector moiety targets a diseased tissue.

33. The conjugate of claim 27, wherein a non-cysteine residue in the CH4 dimerization domain of the first or second fusion protein is replaced with a cysteine residue to create the free cysteine residue.

34. The conjugate of claim 27, wherein i) the CH4 dimerization domain of the first or second fusion protein comprises the amino acid sequence of SEQ ID NO: 28, or ii) the CH4 dimerization domain of the first or second fusion protein comprises an amino acid sequence of SEQ ID NO: 28 that is mutated such that a non-cysteine residue at position Ala4, Val46, Ala51, Ala77, Lys82, Ser95, Gln101, Ala103 or Ser105 is replaced with a cysteine residue that is the site of attachment to the linker.

35. (canceled)

36. The conjugate of claim 27, wherein the first and the second fusion protein each further comprises an M2″ extension of an IgE splice variant located between the CH4 dimerization domain and the C-terminus of the protein.

37. The conjugate of claim 27, wherein the polypeptide spacer sequences of both the first and second fusion protein each comprises an IgG hinge.

38. (canceled)

39. The conjugate of claim 37, wherein the hinge region of at least one of the first and the second fusion protein comprises an amino acid sequence selected from the group consisting of SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, and SEQ ID NO: 59.

40. The conjugate of claim 27, wherein the polypeptide spacer sequences of both the first and the second fusion proteins each comprises 1-200 amino acids, and each is covalently bound to the biological effector moiety and the CH4 dimerization domain.

41. A pharmaceutical composition comprising the conjugate of claim 27 and a pharmaceutically acceptable excipient.

42. A method of killing or inhibiting the proliferation of tumor cells or cancer cells comprising administering to a patient in need thereof an effective amount of the conjugate of claim 27, wherein the dimeric molecular complex of the conjugate has at least one biological effector moiety that comprises an antigen binding site for a tumor-associated antigen and M is a cytotoxic agent or a drug that is a chemotherapeutic agent.

43. A method of imaging a target area of a body in a patient in need thereof comprising administering to a patient a conjugate of claim 27 in an amount sufficient to provide a detectable signal at the target area and detecting the signal, wherein at least one of the biological effector moieties of the first and second fusion proteins targets a specific cell type or tissue in need of imaging and M of the conjugate is a detectable label.

44. A method of making the conjugate of claim 27, comprising reacting:
(I) a dimeric complex that comprises a first and a second fusion protein, wherein each fusion protein comprises from its N to C terminus:
(A) a biological effector moiety selected from the group consisting of:
(1) a single chain antibody;
(2) an Fab fragment;
(3) an extracellular domain of a type I membrane receptor;
(4) a cytokine;
(5) a chemokine;
(6) an enzyme; and
(7) a toxin; wherein at least one of the fusion proteins has a biological effector moiety that is a targeting biological effector moiety selected from the group consisting of (1) a single chain antibody; (2) an Fab fragment; (3) an extracellular domain of a type I membrane receptor; (4) a cytokine; (5) a chemokine; and (6) an enzyme;
(B) a polypeptide spacer sequence; and
(C) a CH4 dimerization domain of an IgE molecule covalently bound to the polypeptide spacer sequence;
wherein the molecular complex comprises at least one disulfide bond between a cysteine residue in the first fusion protein and a cysteine residue in the second fusion protein, and at least one of the fusion proteins comprises one or more free cysteine amino acid residues with
(II) Lₐ-M, wherein if a is 1 then L has a functional group that reacts preferentially with the molecular complex at the free cysteine such that the conjugate is formed and if a is 0 then M has a functional group that reacts preferentially with the molecular complex at the free cysteine such that the conjugate is formed.

45. The method of claim 44, wherein a is 1 and the linker (L) has a maleimide functional group that reacts with the free cysteine to covalently attach the dimeric molecular complex to L-M.
